(19) United States
(12) Patent Application Publication
(10) Pub. No.: US 2004/0199243 A1
(43) Pub. Date: Oct. 7, 2004

(54) Filtering device and method for a venous furcation

(76) Inventor: Ofer Yodfat, Modi'in (IL)

Correspondence Address: Anthony Castorina, G E Ehrlich, Suite 207, 2001 Jefferson Davis Highway, Arlington, VA, 22202, US

(21) Appl. No.: 10/482,954

(22) PCT Filed:
(86) PCT No.: PCT/IL02/00528

(30) Foreign Application Priority Data
Jul. 9, 2001 (IL) 144213

(51) Int. Cl. A61F 2/06

(52) U.S. Cl. 623/1.16; 623/1.15; 623/1.51

(57) ABSTRACT

Implantable blood filtering device (20) and method for filtering embolic material (circles) from blood flowing from at least one of source veins (62) and (68) into the sink vein (64) of a venous furcation (60). Device (20) is an expansible, tubular shaped porous mesh-like element (22) of filaments (24), having first end region (e1) positional in a first source vein of venous furcation (60), second end region (e2) positional in a second source vein or in sink vein (64) of venous furcation (60), and middle filtering zone (F) circumferentially and longitudinally extending between first (e1) and second (e2) end regions, whereby middle filtering zone (F) of element (22) when so positioned in venous furcation (60), filters embolic material from blood passing through pores (26) of middle filtering zone (F), while substantially not disturbing flow of blood through venous furcation (60), thereby preventing embolic material from entering sink vein (64) of venous furcation (60).

### FIELD AND BACKGROUND OF THE INVENTION

The present invention relates to implantable medical devices for filtering embolic material from blood flowing through venous blood vessels, and more particularly, to an implantable blood filtering device and corresponding method for filtering embolic material from blood flowing from at least one source vein into the sink vein of a venous furcation in a subject. The implantable blood filtering device, herein, also referred to as the blood filtering device, is an expansible, tubular shaped porous mesh-like element of filaments, having a first end region positional in a first source vein of the venous furcation, a second end region positional in a second source vein or in the sink vein of the venous furcation, and a middle filtering zone circumferentially and longitudinally extending between the first and second end regions, whereby the middle filtering zone of the element when so positioned in the venous furcation, filters the embolic material from the blood passing through pores of the middle filtering zone, while substantially not disturbing flow of the blood through the venous furcation, thereby preventing the embolic material from entering the sink vein of the venous furcation in the subject.

In the context of the present invention, the term ‘embolic material’ generally refers to the various different types of biological entities, materials, or substances, such as emboli, blood clots, and thrombi, which may be present in blood flowing in the circulatory system of a subject, and which are capable of obstructing and/or preventing blood flow through a blood vessel, thereby leading to various different types of undesirable and serious circulatory and/or other medical conditions in the subject.

A ‘venous furcation’ generally refers to a venous blood vessel featuring a sink (blood receiving or central) vein (branch) which divides or furcates into at least two source (blood supply or side) veins (branches). Exemplary venous furcations are a venous bifurcation, referring to a venous blood vessel featuring a sink (blood receiving or central) vein (branch) which divides or bifurcates into two source (blood supply or side) veins (branches), and, a venous trifurcation, referring to a venous blood vessel featuring a sink (blood receiving or central) vein (branch) which divides or trifurcates into three source (blood supply or side) veins (branches).

Embolic material carried in the blood stream often constitutes serious threats to health and in some instances, to life itself. The elimination, or at least reduction and/or stabilization, of embolic material, and arrest of further migration of embolic material in the circulatory system of a subject, are goals constantly motivating the development by the medical profession of new techniques and devices for this purpose. Although embolic material moving in other portions of the circulatory system can also present serious problems, development of means for preventing embolic material from migrating into the pulmonary circulation from the lower limbs and the vena cava has received primary attention. Embolic material entering the lungs can cause pulmonary embolism (PE) which, if untreated, often leads to death.

Ligation of the vena cava is an early technique first developed in 1930 by DeBakey, for minimizing movement of embolic material therein, with collateral circulation relied upon for providing adequate venous return of blood to the heart. From this procedure, which involves major abdominal surgery, the development of methods to prevent embolic material from entering the lungs progressed through many technological stages and advances up to the present day use of intravascular filters, also known as blood vessel filters.

Intravascular filters function by preventing relatively large sized embolic materials, particularly, blood clots and thrombi, from traveling, typically from leg veins, through the inferior vena cava, to the heart and into the lungs. Typically, intravascular filters are surgically introduced into a blood vessel by cutting down to and then into a vein, using surgical blades. This surgical procedure ordinarily requires two teams of surgeons and it is not uncommon for the procedure to take up to two hours.

In recent years, techniques have been developed and implemented for percutaneously inserting certain types of intravascular filters. The advantages of these techniques include reduced trauma and shortened surgical time. According to two recent articles reviewing vena cava (caval) filters, Streiff, Michael B., Vena caval filters: a comprehensive review, in Blood 95, Number 12, 15 Jun. 2000, 3669-3677, and, Procter, et al., In Vivo Evaluation of Vena Caval Filters: Can Function Be Linked to Design Characteristics?, in Cardiovasc Intervent Radiol, 2000, 23, 460-465, five different vena cava filters are presently in use in the United States. These filters are shown in FIG. 1, which is taken from the Streiff article. The five vena cava filters are illustrated in FIG. 1 as follows: (A) the stainless steel Greenfield filter, (B) the modified-hook titanium Greenfield filter, (C) the bird's nest filter, (D) the Simon nitinol filter, and, (E) the Vena Tech Filter.

As illustrated in FIG. 1, there are two general types of vena cava filters. The first general type of vena cava filter, (A), (B), and (E), is typically formed of fine wire legs attached to a head or nose cone. The wire legs have a conical aspect in order to channel embolic material toward the center of the filter, to be entrapped by the nose cone near the apex of the filter. The second general type of vena cava filter, (C) and (D) in FIG. 1, consists of a wire mesh inserted into and anchored in the interior vena cava. Depending on age, nature, and geometrical characteristics, the embolic material, particularly, blood clots and thrombi, may permanently remain in the filter or may be lysed using a fibrinolysis technique.

According to in vitro studies, the clot-trapping rate, that is, the number of blood clots trapped per total number of blood clots entering the filter, is in direct relation to the size of the blood clot, that is, the larger the size of the blood clot, the higher the trapping rate. Most of the above mentioned vena cava filters do not trap blood clots smaller than about 1.5 mm in diameter, but trap nearly 100% of blood clots larger than about 4-5 mm in diameter, as described by Jaeger H. J., et. al., A physiologic in vitro model of the inferior vena cava with a computer-controlled flow system for testing of inferior vena cava filters, in Invest Radiol 1997 Sepember, 32(9), 511-22; Simon M., et. al., Comparative evaluation of clinically available inferior vena cava filters with an in vitro physiologic simulation of the vena cava, in Radiology, 1993 December, 189(3), 769-74; Jager, et. al., In vitro model for the evaluation of inferior vena cava filters: effect of experimental parameters on thrombus-capturing efficacy of the Vene Tech-LGM filter, in J Vasc Interv Radiol, 1998 March-April, 9(2), 295-305.

One of the early blood clot filters of the first general type described above is that of Kimmell disclosed in U.S. Pat. No. 3,952,747. The Kimmell filter has a plurality of stainless steel wire legs extending from a large head. The legs are arranged in a conical aspect, wherein each leg is bent to form a number of linear segments generally tangent about the conical aspect, for increasing the filtering effect. The end, not attached to the head, of each leg is bent to form a hook, which is designed to engage the wall of the vessel and anchor the device. When the filter is inserted into a blood vessel, the head and the apex of the cone are positioned downstream in the blood flow. The remote ends, not attached to the head, of the legs are positioned upstream in the blood flow and are engaged with the vessel wall. The Kimmell disclosure also teaches of a system for expanding and implanting the device in situ.

Because of the relatively large diameter of the Kimmell device in its collapsed position, it can not be introduced into the blood vessel using conventional percutaneous catheterization techniques, whereby it is necessary to perform a venotomy. Guiding the device to and releasing it at the desired location is complicated and time consuming. In addition, the hooks of the legs may damage or even puncture the vessel walls, and/or improperly anchor the device, and/or insufficiently anchor the device. Improper anchoring of the device results in the device migrating and/or tilting with respect to the axis of the vein, thereby reducing the effectiveness of the device for filtering embolic material from the blood.

Improvements on the Kimmell design are found in, for example, U.S. Pat. No. 5,059,205, assigned to the distributor of the Greenfield filter devices. In a later development of the same basic idea, in U.S. Pat. No. 6,214,025, issued to Thistle et al., there is disclosed a blood filter in which the legs are replaced by a generally cylindrical radially expansible anchoring region to which is attached a conical filtering region. In U.S. Pat. No. 4,425,908, issued to Simon, there is disclosed the basic design of the Simon nitinol filter.

One of the earliest intravascular filters of the second general type described above, for entrapping and arresting of embolic material is disclosed in U.S. Pat. No. 3,540,431, issued to Mobin-Uddin et al. The Mobin-Uddin filter is an umbrella type structure which includes a plurality of expanding struts or ribs which carry points at the divergent ends thereof which impale or engage the vessel wall when the filter is in its implanted expanded state. This device is introduced through a small incision in the jugular vein and passed through the heart for positioning in the inferior vena cava. The Mobin-Uddin filter is associated with problems relating to its migration.

One version of the present day bird's nest type blood filter is disclosed in U.S. Pat. No. 4,494,531, issued to Gianturco. The disclosed blood filter is comprised of a number of strands of shaped memory wire which are interconnected and wadded together to form a curly wire mesh. The strands can be straightened for insertion into the lumen. When released, the filter takes roughly the shape shown in FIG. 1 (C). The filter includes a number of projections, which serve as anchoring points.

In U.S. Pat. No. 5,976,172, issued to Homsma et al., there is disclosed a retractable temporary vena cava filter, and in U.S. Pat. No. 6,099,549, issued to Bosma et al., there is disclosed a vascular filter for controllable release. Each of these implantable blood filters has highly specialized structural features for supposedly expediting and enabling insertion, deployment, and retraction or removal, of the filter from a vessel.

Main categories of limitations, shortcomings, and problems associated with the use of veneous filters are as follows: (a) Mechanics, relating to filter migration; damage to, or even puncturing of, the wall of the vein by the filter anchoring hooks; tilting of the filter with respect to the long axis of the vein, resulting in reduced filtering efficiency; and, fracture of the filter device; (b) Filter Size, relating to the relatively large dimensions of the filter openings or pores, which result in trapping only large sized embolic material; increasing of the dimensions of the filter openings or pores as the diameter of the inferior vena cava increases, resulting in lager spaces between the filter legs; and, (c) Insertion or Deployment, relating to the overall diameter of the collapsed filter requiring use of relatively large diameter insertion catheters; and, (d) Thrombogenicity and unfavorable hemodynamics flow profile.

The mechanical problems listed above have been reduced in presently used blood filter models, however they still occur in a significant percentage of cases. In particular, migration, 1.9-12.8%; penetration of the IVC wall, 1%; significant tilting 1-12.4%; fracture of the device, 1.2-2.8%, as reported in the previously cited Steiff article.

As a gold standard, the diagnosis of pulmonary emboli is made by angiography. A clot is present if there is observed either a constant intraluminal filling defect, or, an abrupt cut-off in vessels larger than 2.5 mm in diameter, as described by Wells P S, et. al., Use of a clinical model for safe management of patients with suspected pulmonary embolism, in Ann Intern Med 1998, Dec. 15, 129(12), 997-1005.

According to the three in vitro studies cited above, currently used blood filters trap approximately 100% of clots 4-5 mm or larger in diameter, but only 59% of clots 2.5-4 mm in diameter. Since it is well known in medical literature that small clots are also major causes of pulmonary hypertension, it is clear that there is still further need to improve the quality of filtering blood in subjects.

Concerning the size of insertion or deployment devices, the original Greenfield filter uses a 29F insertion catheter, later models have reduced the diameter to 14F, which is also that used for the bird's nest type blood filter. The Simon-Nitinol filter has the smallest diameter, requiring a 9F insertion catheter. Reducing the size of the insertion catheter facilitates the insertion procedure and also reduces side effects at the insertion site. These side effects include local hematoma, postphebitic syndrome, insertion site thrombus formation, and femoral vein puncture.

Currently, an alternative treatment of pulmonary embolism is the use of anticoagulation drugs. Although anticoagulents cannot be given to certain groups of patients, for example, cancer patients, many of the elderly, major trauma cases, etc., they are generally used, whenever possible, as the treatment of choice, and venous cava filters are mostly used only when the drug approach is not possible. This is partly a result of resistance or anxiety to using intravascular filters due to collective memory of the failures and difficulties of insertion associated with the early blood filters. Cost effectiveness is also cited as a reason for choice of treatment type, although long-term drug treatment can be at least as costly as the procedure for inserting an intravascular filter. There appears to be a trend to increase the use of intravascular filters, for example, as reported in an internet article published by the American College of Chest Physicians in the framework of its PCCU ONLINE program: Robert J. Schilz and Joel Worth, Lesson 3, Volume 14—Use of Vene Cava Filters in the Management of Veneous Thromboembolic Disease, www.CHESTNET.ORG/EDUCATION/PCCU/VOL14/LESSON 03 html, posted Oct. 27, 1999. This trend would be accelerated if the physical dimensions, stability, and filtering ability of venous cava filters could be improved.

A study by Decousus H. et.al., A clinical trial of vena cava filters in the prevention of pulmonary embolism in patients with proximal deep-vein thrombosis, in the NEJM 1998, 338(7), 409, that investigated the efficacy of the new generation of inferior venal cava filters (IVCFs), showed that after two years there was no significant difference in the incidence of pulmonary embolism between the group in which IVCFs were implanted and a group that was treated only with anticoagulants. It was also found that there was a higher incidence of deep venous thrombosis (DVT) in the group that received the filters. This study, in which the authors assumed, for the first time, that the filters themselves are a thrombogenic cause, has been followed by others that have shown high incidence of thrombi at the filter implantation sites.

Factors affecting thrombus formation of these biocompatible devices that are placed inside blood vessels are divided into three major catagories: (1) fluid mechanical factors, (2) vascular factors, and, (3) blood related factors, as described by Turitto V T, et. al., Cells and aggregates at surfaces, in Ann N Y Acad Sci 1987, 516, 453-467.

Studies by Decousus H, et.al., A clinical trial of vena cava filters in the prevention of pulmonary embolism in patients with proximal deep-vein thrombosis. Prevention du Risque d'Embolie Pulmonaire par Interruption Cave Study Group., in NEJM 1998 Feb, 12, 238 and 409-15; Wittenberg G., et. al., Long-term results of vena cava filters: experiences with the LGM and the Titanium Greenfield devices, in Cardiovasc Intervent Radiol 1998 May-June, 21(3), 225-9; Helmberger T., et. al., Vena cava filter. Indications, complications, clinical evaluation, in Radiologe 1998 July, 38(7), 614-23; Tardy B., et. al., Symptomatic inferior vena cava filter thrombosis: clinical study of 30 consecutive cases, in Eur Respir J 1996 October, 9(10), 2012-6, involved directly imaging IVC filters by computerized tomographic (CT) scan and/or duplex ultrasonography, in an attempt to determine long term patency, reported rates of significant thrombosis/occlusion ranging from 3.5-31%, depending upon the type of IVC filter device.

Over the last two decades, substantial evidence has been accumulated suggesting that fluid mechanical factors can be extremely important in modulation of the molecular mechanisms of platelet and thrombi formation. The fluid mechanical factors affecting thrombus formation by intraluminal devices are described by three major parameters: (i) the shear stress caused by the blood flowing through the device, (ii) the exposure time of the blood to the device, and (iii) the Reynolds number, Re, of the blood flowing through the device. The Reynolds number is dependent on the cross section perimeter of the elements of which the device is constructed. The lower the Reynolds number, the smaller the recirculation region of the blood stream after passing the structural element of the device.

The concept of shear induced platelet activation has been experimentally and theoretically investigated since the mid 1970s, as disclosed by Colantuoni G, et. al., The response of human platelets to shear stress at short exposure times, in Trans Am Soc Artif Int Organs 1977, 23, 626-31, and, by Ramstack J M, et. al., Shear-induced activation of platelets, in J. Biomech 1979, 12(2), 113-25.

Deposition of platelets onto artificial surfaces tends to increase with increasing shear, as taught by Goodman S L, et. al., In vitro vs. Ex vivo platelet deposition on polymer services, in Scan Electron Microsc 1984 (Part 1), 279-90, and, thrombus formation is preceded by platelet activation, in areas of high shear flow, and is followed by platelet deposition onto the vessel wall, in areas of stasis and recirculation, as taught by Aarts P. A., et. al., Blood platelets are concentrated near the wall and red blood cells in the center of flowing blood, in Arteriosclerosis 1988 November-December, 8(6), 819-24.

Shear stress of at least 50 dynes/cm² triggers platelet activation, causing release of granule contents and elicit platelet aggregation. Shear stress higher than about 100 dynes/cm² results in the appearance of non-storage nucleotides and other cellular contents, including cell lysis. Thrombogenicity is influenced by accumulation of the shear stress and the exposure time of the blood to the constructional elements, that is, the filaments, fibers, or strands, of the device. Reducing the cross section perimeter of the constructional elements of the device shortens the resident time of the high shear stress region, therefore reducing the amount of thrombogenicity.

The Reynolds number, Re, is determined from the equation Re=U*d*q/υ, where q/υ is the kinematic viscocity (3.5*10⁻⁶ m²/sec for blood in the inferior vena cava), U is the mean blood velocity (10 cm/sec in the inferior vena cava), and d is the diameter of circular or round filament, fiber, wire, or strand, type of structural elements of the device. For each device there is defined an average Reynolds number, Reₐᵥₑ, which is based on the average diameter of all the structural elements of the device. The size of the recirculation region is directly related to the Reynolds number of the structural elements. Therefore, the lower the value of Rₐᵥₑ, the lower the activation of the coagulation system and the lower the thrombogenicity of the device.

Since the mean blood velocity, U, and the kinematic viscosity, q/υ, of the blood are the same for all devices implanted in the inferior vena cava, the value of the Reynolds number, Re, is directly proportional to the cross section perimeter or diameter of the structural elements of the device. Vena cava filters currently in widespread use have structural elements consisting of wires having diameters ranging from 0.18 mm (bird's nest type filter) to 0.45 mm (Greenfield type filter) and lengths between 3 cm (Simon Nitinol type filter) and 7 cm (bird's nest type filter). Wires of these dimensions are necessary to provide sufficient strength and anchoring forces, especially in the case of filters designed for placement in venae cavae having relatively large dimensions.

Taking into consideration the above discussion of the factors affecting thrombus formation, the strong thrombogenic effect of currently used vena cava filters can be easily understood. Observed phenomena of formation of thrombi on vein walls at the site of implantation and on the filter struts, occlusion of the filters, reduction in patency, and cases of recurrent DVT, are all consequences of the relatively large sizes of the wires used as the structural elements for constructing the filters taught about in the prior art.

There is thus a need for, and it would be highly advantageous to have an implantable blood filtering device and corresponding method for filtering embolic material from blood flowing from at least one source vein into the sink vein of a venous furcation in a subject, while substantially not disturbing flow of the blood through the venous furcation, thereby preventing the embolic material from entering the sink vein of the venous furcation in the subject. Moreover, there is a strong need for such an invention which overcomes all of the above described limitations, shortcomings, and problems associated with the use of prior art intraluminal, intravascular, blood filter devices and techniques.

### SUMMARY OF THE INVENTION

The present invention relates to an implantable blood filtering device and corresponding method for filtering embolic material from blood flowing from at least one source vein into the sink vein of a venous furcation in a subject. The implantable blood filtering device is an expansible, tubular shaped porous mesh-like element of filaments, having a first end region positional in a first source vein of the venous furcation, a second end region positional in a second source vein or in the sink vein of the venous furcation, and a middle filtering zone circumferentially and longitudinally extending between the first and second end regions, whereby the middle filtering zone of the porous mesh-like element when so positioned in the venous furcation, filters the embolic material from the blood passing through pores of the middle filtering zone, while substantially not disturbing flow of the blood through the venous furcation, thereby preventing the embolic material from entering the sink vein of the venous furcation in the subject.

The expansible, tubular shaped porous mesh-like element has a variable geometrical configuration or construction characterized by a combination of critical ranges of values of dimensional characteristics, for optimally filtering the embolic material from the blood passing through pores of the middle filtering zone, and maintaining a deployed implanted expanded position in the venous furcation, while substantially not disturbing flow of the blood through the venous furcation, thereby highly effectively preventing the embolic material from entering the sink vein of the venous furcation and from migrating downstream therefrom in the circulatory system of the subject.

Thus, according to a first aspect of the present invention, there is provided an implantable blood filtering device for implantation in a venous furcation of two source veins into a sink vein to filter embolic material from the blood in one of said source veins before flowing into said sink vein; the device comprising a tubular-shaped porous structure having a first end region configured and dimensioned for anchoring in one of the veins at the venous furcation; a second end region configured and dimensioned for anchoring in another of the veins at the venous furcation; and a middle filtering zone between the first and second end regions; the middle filtering zone having pores configured and dimensioned so as to be effective, when the first and second end regions of the tubular-shaped porous structure are anchored in their respective veins, to filter the embolic material in the blood flowing in the one source vein before entering the sink vein.

According to another aspect of the present invention, there is provided a method for filtering embolic material in a source vein from flowing into a sink vein at a venous furcation of said sink vein with said source vein and at least one other source vein, comprising: providing an implantable blood filtering device comprising a tubular-shaped porous structure having a first end region configured and dimensioned for anchoring in one of the veins at the venous furcation; a second end region configured and dimensioned for anchoring in another of the veins at the venous furcation; and a middle filtering zone between the first and second end regions; the middle filtering zone having pores configured and dimensioned so as to be effective, when the first and second end regions of the tubular-shaped porous structure are anchored in their respective veins, to filter the embolic material in the blood flowing in the one source vein before entering the sink vein; and implanting the filtering device into the venous furcation.

According to another aspect of the present invention, there is provided a method for preventing and/or treating the occurrence of a condition associated with embolic material in blood flowing from at least one source vein into the sink vein of a venous furcation in a subject, featuring the steps of: (a) providing an implantable blood filtering device comprising an expansible, tubular shaped porous mesh-like element of filaments, having a first end region positional in a first source vein of the venous furcation, a second end region positional in a second source vein or in the sink vein of the venous furcation, and a middle filtering zone circumferentially and longitudinally extending between the first and second end regions; and (b) implanting and deploying the implantable blood filtering device in the venous furcation, whereby the middle filtering zone of the mesh-like element when so positioned in the venous furcation, filters the embolic material from the blood passing through pores of the middle filtering zone, while substantially not disturbing flow of the blood through the venous furcation, thereby preventing the embolic material from entering the sink vein of the venous furcation of the subject.

### BRIEF DESCRIPTION OF THE DRAWINGS

The present invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the present invention. In this regard, no attempt is made to show structural details of the present invention in more detail than is necessary for a fundamental understanding of the invention, the description taken with the drawings making apparent to those skilled in the art how the several forms of the invention may be embodied in practice. In the drawings:

FIG. 1 (prior art) is a schematic diagram illustrating different types of prior art vena cava intravascular filters;

FIG. 2A is a schematic diagram illustrating an exemplary preferred embodiment of the implantable blood filtering device, in accordance with the present invention;

FIG. 2B is a schematic diagram illustrating an enlarged view of a small portion of the exemplary preferred embodiment of the implantable blood filtering device of FIG. 2A, in accordance with the present invention;

FIG. 3A is a schematic diagram illustrating a first alternative type of deployment of the exemplary preferred embodiment of the implantable blood filtering device of FIG. 2A, in a venous bifurcation type of venous furcation in a subject, in accordance with the present invention;

FIG. 3B is a schematic diagram illustrating a second alternative type of deployment of the exemplary preferred embodiment of the implantable blood filtering device of FIG. 2A, in a venous bifurcation type of venous furcation in a subject, in accordance with the present invention;

FIG. 3C is a schematic diagram illustrating a third alternative type of deployment of the exemplary preferred embodiment of the implantable blood filtering device of FIG. 2A, in a venous bifurcation type of venous furcation in a subject, in accordance with the present invention;

FIG. 4 is a schematic diagram illustrating an exemplary preferred embodiment of a first alternative form of the implantable blood filtering device of FIGS. 2A and 2B, wherein the geometrical configuration or construction is characterized by a variable inter-region structural profile, in accordance with the present invention;

FIG. 5 is a schematic diagram illustrating a structural/functional blood filtering device implementation problem commonly existing in blood vessels which are part of a venous furcation, which is prevented by using the second, third, or fourth alternative form of implantable blood filtering device of FIGS. 2A-2B, illustrated in FIGS. 6-8, respectively, in accordance with the present invention.

FIG. 6 is a schematic diagram illustrating an exemplary preferred embodiment of a second alternative form of the implantable blood filtering device of FIGS. 2A and 2B, wherein the geometrical configuration or construction is characterized by a variable inter-region structural profile, in accordance with the present invention;

FIG. 7 is a schematic diagram illustrating an exemplary preferred embodiment of a third alternative form of the implantable blood filtering device of FIGS. 2A and 2B, wherein the geometrical configuration or construction is characterized by a variable inter-region structural profile and by variable intra-region structural profiles, in accordance with the present invention;

FIG. 8 is a schematic diagram illustrating an exemplary preferred embodiment of a fourth alternative form of the implantable blood filtering device of FIGS. 2A and 2B, wherein the geometrical configuration or construction is characterized by a variable inter-region structural profile and by a variable intra-region structural profile, in accordance with the present invention; and

FIG. 9 is a schematic diagram illustrating exemplary venous bifurcation types of venous furcations in the circulatory system of a subject, applicable to deploying the exemplary preferred embodiments of the implantable blood filtering device, according to the previously described three alternative types of deployment illustrated in FIGS. 3A-3C, and in FIG. 5, in accordance with the present invention.

### DESCRIPTION OF THE PREFERRED EMBODIMENTS

The present invention relates to an implantable blood filtering device and corresponding method for filtering embolic material from blood flowing from at least one source vein into the sink vein of a venous furcation in a subject. The implantable blood filtering device, herein, also referred to as the blood filtering device, is an expansible, tubular shaped porous mesh-like element, herein, also referred to as a mesh-like element, of filaments, having a first end region positional in a first source vein of the venous furcation, a second end region positional in a second source vein or in the sink vein of the venous furcation, and a middle filtering zone circumferentially and longitudinally extending between the first and second end regions, whereby the middle filtering zone of the mesh-like element when so positioned in the venous furcation, filters the embolic material from the blood passing through pores of the middle filtering zone, while substantially not disturbing flow of the blood through the venous furcation, thereby preventing the embolic material from entering the sink vein of the venous furcation in the subject.

The expansible, tubular shaped porous mesh-like element has a variable geometrical configuration or construction characterized by two types of structural profiles, which are, (1) an ‘inter-region’ structural profile and (2) ‘intra-region’ structural profiles, determined by a combination of critical ranges of values of dimensional characteristics, for optimally filtering the embolic material from the blood passing through pores of the middle filtering zone, and maintaining a deployed implanted expanded position in the venous furcation, while substantially not disturbing flow of the blood through the venous furcation, thereby highly effectively preventing the embolic material from entering the sink vein of the venous furcation and from migrating downstream therefrom in the circulatory system of the subject.

Herein, the terms ‘embolic material’ and ‘venous furcation’ are referred to and used in a manner consistent with their respective denotations in the field of medicine. The term ‘embolic material’ generally refers to the various different types of biological entities, materials, or substances, such as emboli, blood clots, and thrombi, which may be present in blood flowing in the circulatory system of a subject, and which are capable of obstructing and/or preventing blood flow through a blood vessel, thereby leading to various different types of undesirable and serious circulatory and/or other medical conditions in the subject.

A ‘venous furcation’ generally refers to a venous blood vessel featuring a sink (blood receiving or central) vein (branch) which divides or furcates into at least two source (blood supply or side) veins (branches). Exemplary venous furcations are a venous bifurcation, referring to a venous blood vessel featuring a sink (blood receiving or central) vein (branch) which divides or bifurcates into two source (blood supply or side) veins (branches), and, a venous trifurcation, referring to a venous blood vessel featuring a sink (blood receiving or central) vein (branch) which divides or trifurcates into three source (blood supply or side) veins (branches). In the context of the present invention, in a non-limiting manner, a venous furcation primarily refers to a venous bifurcation, however, the invention is equally applicable to a venous trifurcation.

Hereinafter, for the purpose of brevity and clarity of description, the phrases ‘sink vein’, ‘source vein’, and ‘source veins’, are used when referring to a venous furcation for describing the present invention. However, it is to be clearly understood that the phrase ‘sink vein’ is synonymous with the synonymous phrases ‘blood receiving vein’, ‘blood receiving branch’, ‘central vein’, and ‘central branch’, of the venous furcation, and, that the phrase ‘source vein’ is synonymous with the synonymous phrases ‘blood supply vein’, ‘blood supply branch’, ‘side vein’, and ‘side branch’, of the venous furcation.

For completeness of description, a source vein of a first venous furcation may be structured and function additionally as a sink vein of a second venous furcation. Equivalently stated, a sink vein of a first venous furcation may be structured and function additionally as a source vein of a second venous furcation. Consistent with the herein described structure and function of the blood filtering device of the present invention, the blood filtering device is deployed and operates inside a venous furcation whereby the direction of the blood flowing in the venous furcation is from and through each of the at least two source veins toward and into the sink vein of the venous furcation.

A first exemplary specific application of the present invention is whereby the blood filtering device filters embolic material from blood flowing from and through the right and/or left common iliac veins (source veins) towards the inferior vena cava vein (sink vein) of the bifurcation of the inferior vena cava vein, thereby preventing the embolic material from entering the inferior vena cava vein (sink vein) and from migrating downstream therefrom in the circulatory system of a subject.

A second exemplary specific application of the present invention is whereby the blood filtering device filters embolic material from blood flowing from and through the internal and/or external iliac veins (source veins) towards a common iliac vein (sink vein) of the bifurcation of a common iliac vein, in particular, the right or left common iliac vein, thereby preventing the embolic material from entering the common iliac vein (sink vein) and from migrating downstream therefrom in the circulatory system of a subject.

Main aspects of novelty and inventiveness of the present invention, are that the implantable blood filtering device is designed and constructed specifically for optimally filtering the embolic material from the blood passing through pores of the middle filtering zone, and maintaining a deployed implanted expanded position in the venous furcation, while substantially not disturbing flow of the blood through the venous furcation, thereby highly effectively preventing the embolic material from entering the sink vein of the venous furcation and from migrating downstream therefrom in the circulatory system of the subject.

This is accomplished by geometrically constructing or configuring the expansible, tubular shaped porous mesh-like element of the blood filtering device according to two types of structural profiles, which are, (1) an inter-region structural profile and (2) intra-region structural profiles, determined by a unique combination of critical ranges of values of dimensional characteristics in the implanted expanded state, according to desired and/or required placement, configuration, and operation of the mesh-like element inside the venous furcation.

According to actual requirements of implementation, in specific forms of the preferred embodiment of the blood filtering device of the present invention, the geometrical configuration or construction of the mesh-like element, in the implanted expanded state, is characterized by (1) an inter-region structural profile, whereby values of at least one dimensional characteristic from region to region of at least two of the three regions being the first end region, the second end region, and the middle filtering zone, are either constant or vary, that is, are the same or different, and, characterized by (2) intra-region structural profiles, whereby values of at least one dimensional characteristic within at least one region of the three regions, that is, within one or both of the first and second end regions, and/or, within the middle filtering zone, are either constant or vary as a function of longitudinal length within each corresponding region along a longitudinal axis of the mesh-like element in the implanted expanded state. Regarding (2) intra-region structural profiles, the variation is either a continuous variation, or, a non-continuous or discrete variation as a function of longitudinal length within each corresponding region along a longitudinal axis of the mesh-like element in the implanted expanded state.

Particular aspects of novelty and inventiveness of the present invention relate to the unique and variable positioning and anchoring of the expansible, tubular shaped porous mesh-like element inside the venous furcation of a subject. The first end region of the mesh-like element is positional in a first source vein of the venous furcation and the second end region is positional in either a second source vein or in the sink vein of the venous furcation. When the mesh-like element is so positioned in the venous furcation, and maintains, by self-anchoring to inner wall regions of the venous furcation, a deployed implanted expanded position in the venous furcation, the middle filtering zone circumferentially and longitudinally extending between the first and second end regions, filters the embolic material from the blood passing through pores of the middle filtering zone, while substantially not disturbing flow of the blood through the venous furcation.

Dimensional characteristics of the expansible, tubular shaped porous mesh-like element of the implantable blood filtering device in the implanted expanded state, having critical ranges of values, are: (i) the cross section perimeter of the mesh-like element filaments, (ii) the length of a side of each opening or pore formed between the mesh-like element filaments in the implanted expanded state, (iii) the number of filaments of the mesh-like element, (iv) the angle of the crossed or overlapped mesh-like element filaments in the implanted expanded state, referring to either the right angle, 90°, between two adjacent sides of a square shaped opening or pore, or, referring to the obtuse angle, between 90° and 180°, between two adjacent sides of a non-square, parallelogram, shaped, opening or pore, formed between the crossed or overlapped mesh-like element filaments in the implanted expanded state, (v) the pitch of turnings of mesh-like element filaments in the implanted expanded state, referring to the distance along a same longitudinal axis of the mesh-like element, between two corresponding points located on adjacent turnings of mesh-like element filaments in the implanted expanded state, (vi) the porosity index of the mesh-like element in the implanted expanded state, (vii) the diameter of the mesh-like element in the implanted expanded state, and, (viii) the luminal length of the mesh-like element in the implanted expanded state. Dimensional characteristics (i)-(viii) are for the mesh-like element ‘in the implanted expanded state’, that is, for the mesh-like element in the expanded state positioned, implanted, and deployed inside the venous furcation of the subject.

The geometrical configuration or construction of the mesh-like element is characterized by an inter-region structural profile, whereby values of at least one of above listed dimensional characteristics (i)-(viii) from region to region of at least two of the three regions being the first end region, the second end region, and the middle filtering zone, are either constant or vary, that is, are the same or different. The geometrical configuration or construction of the mesh-like element is additionally characterized by intra-region structural profiles, whereby values of at least one of above listed dimensional characteristics (i)-(viii) within at least one region of the three regions, that is, within one or both of the first and second end regions, and/or, within the middle filtering zone, are either constant or vary as a function of longitudinal length within each corresponding region along a longitudinal axis of the mesh-like element in the implanted expanded state, where the variation is either a continuous variation, or, a non-continuous or discrete variation. Preferred critical ranges of values of each of these dimensional characteristics used in combination for geometrically configuring the expansible, tubular shaped mesh-like element of the blood filtering device of the present invention, are provided and described in detail below.

Based upon the above indicated main aspect of novelty and inventiveness, the present invention successfully overcomes the limitations, shortcomings, and associated problems, and widens the scope, of presently known intraluminal, intravascular, blood filtering devices and techniques, for application to preventing embolic material from entering the central branch of a vascular bifurcation and from migrating downstream therefrom in the circulatory system of a subject.

In particular, implementation of the present invention successfully overcomes the limitations, shortcomings, and problems associated with the use of prior art intraluminal, intravascular, blood filters, with regard to the three main categories of (a) Mechanics, relating to filter migration; damage to, or even puncturing of, the wall of a blood vessel by filter anchoring hooks; tilting of the filter with respect to the long axis of a blood vessel, resulting in reduced filtering efficiency; and, fracture of the filter device; (b) Filter Size, relating to the relatively large dimensions of the filter openings or pores, which result in trapping only large sized embolic material; increasing of the dimensions of the filter openings or pores as the diameter of the blood vessel increases, resulting in lager spaces between the filter legs; (c) Insertion or Deployment, relating to the relatively large overall diameter of the collapsed filter requiring use of a correspondingly relatively large diameter insertion catheter; and, (d) Thrombogenicity and unfavorable hemodynamics flow profile caused by the presence of relatively large diameter filaments, strands, or fibers of the filtering device.

The implantable blood filtering device of the present invention is capable of filtering, by way of trapping or capturing, and thereby preventing passage of, embolic material having sizes significantly smaller than embolic material currently trapped by prior art intraluminal, intravascular, blood filters. Insertion or deployment of the implantable blood filtering device of the present invention involves using an insertion catheter having a much smaller diameter than is required by prior art intraluminal, intravascular, blood filters. Moreover, the implantable blood filtering device and corresponding method thereof, of the present invention, provide an attractive alternative to anticoagulant drug treatments. Additional benefits and advantages of the present invention are apparent in the following illustrative description.

It is to be understood that the invention is not limited in its application to the details of construction, arrangement, and, composition, of the implantable blood filtering device, or, to the details of the order or sequence of steps of the corresponding method of implementing thereof, set forth in the following description, drawings, or examples. The present invention is capable of other embodiments or of being practiced or carried out in various ways. Also, it is to be understood that the phraseology, terminology, and, notation, employed herein are for the purpose of description and should not be regarded as limiting.

For example, herein, the term ‘mesh-like’ is used throughout the disclosure as a descriptor for further describing and clarifying the geometrical configuration or construction of the expansible, tubular shaped porous element of the implantable blood filtering device, and alternative embodiments thereof, of the present invention. In the context of the present invention, the term ‘mesh-like’ denotes a net or network of crossed or overlapped filaments, fibers, wires, or strands, used for geometrically configuring or constructing the expansible, tubular shaped porous mesh-like element of the implantable blood filtering device, and alternative embodiments thereof, of the present invention. It is to be fully understood that the term ‘mesh-like’ generally refers to synonymous, directly related, alternative, and/or more specific or limiting descriptors such as, but not limited to, braided, plaited, interwoven, interweaved, woven, weaved, interlaced, and knitted, whereby each of these terms may equivalently, relatedly, alternatively, or more specifically, be used as an appropriate descriptor for further describing and clarifying the geometrical configuration or construction of the expansible, tubular shaped porous mesh-like element of the implantable blood filtering device, and alternative embodiments thereof, of the present invention.

Structure and function of the implantable blood filtering device and corresponding method for filtering embolic material from blood flowing from at least one source vein into the sink vein of a venous furcation in a subject, according to the present invention, are better understood with reference to the following description and accompanying drawings. Throughout the following description and accompanying drawings, like reference numbers refer to like elements.

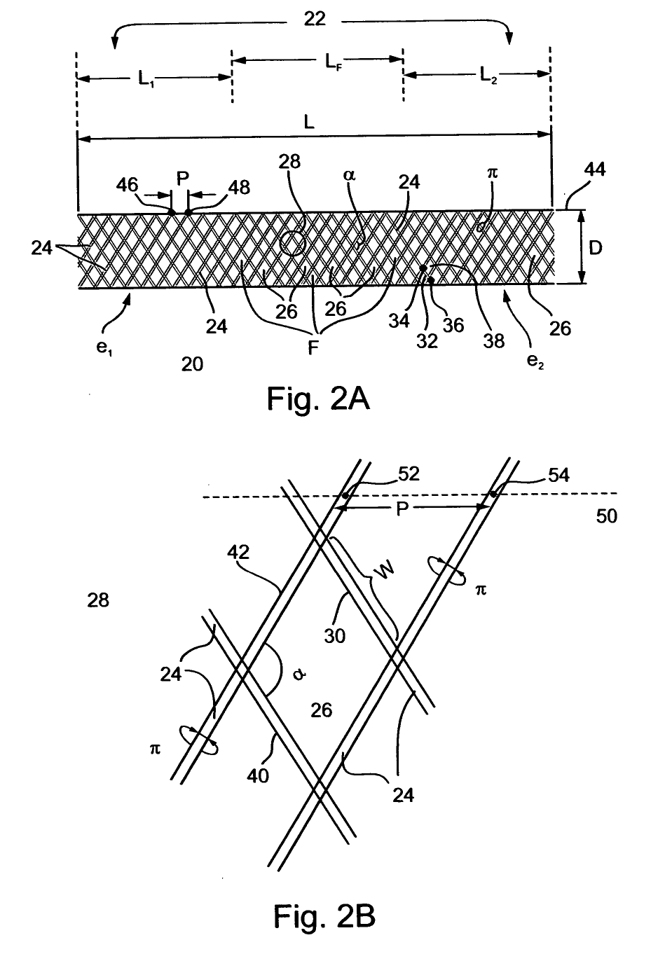

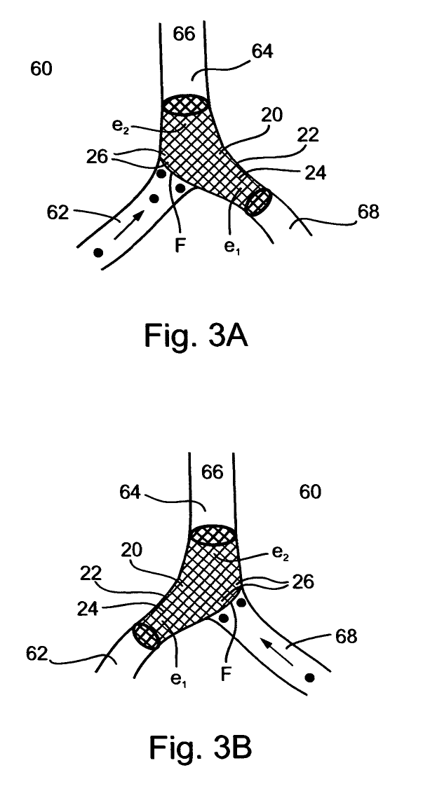

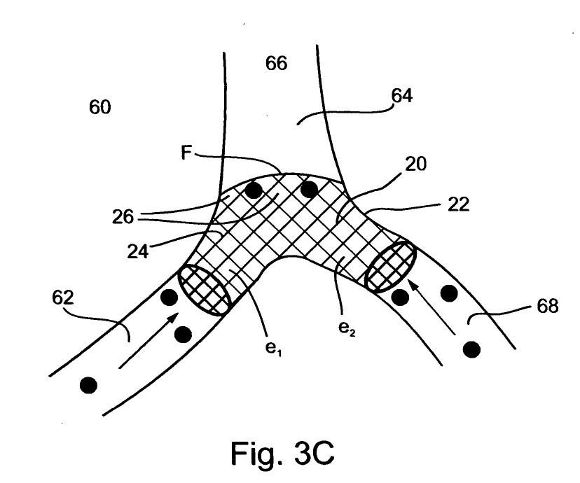

For the purpose of assisting in understanding description of the structure and function of the exemplary preferred embodiment of the implantable blood filtering device of the present invention, brief reference only is herein made to FIGS. 3A-3C, schematic diagrams illustrating three alternative types of positioning and deployment of the exemplary preferred embodiment of the implantable blood filtering device of FIGS. 2A-2B in a venous furcation, where the venous furcation is, for illustrative example, a venous bifurcation. Detailed description of FIGS. 3A-3C, in terms of describing the method of the present invention, are provided hereinafter description of the structure and function of the exemplary preferred embodiment of the implantable blood filtering device of the present invention.

Referring now to the drawings, FIG. 2A is a schematic diagram illustrating an exemplary preferred embodiment of the implantable blood filtering device of the present invention, herein, for brevity, generally referred to as blood filtering device 20. Blood filtering device 20 is an expansible, tubular shaped porous element 22, having a mesh-like geometrical configuration or construction, herein, also referred to as expansible, tubular shaped porous mesh-like element 22, and for brevity, equivalently referred to as mesh-like element 22, formed from filaments, fibers, wires, or strands 24, herein, generally referred to as mesh-like element filaments 24, and for brevity, equivalently, generally referred to as filaments 24. Expansible, tubular shaped porous mesh-like element 22 has openings or pores 26, formed and located in between adjacent mesh-like filaments 24, and, circumferentially and longitudinally extending around and along the entirety of mesh-like element 22.

As previously indicated, herein, the term ‘mesh-like’ is used throughout the disclosure as a descriptor for further describing and clarifying the geometrical configuration or construction of expansible, tubular shaped porous element 22 of implantable blood filtering device 20, and alternative embodiments thereof, of the present invention. In the context of the present invention, the term ‘mesh-like’ denotes a net or network of crossed or overlapped filaments, fibers, wires, or strands 24, used for geometrically configuring or constructing expansible, tubular shaped porous mesh-like element 22 of implantable blood filtering device 20, and alternative embodiments thereof, of the present invention. It is to be fully understood that the term ‘mesh-like’ generally refers to synonymous, directly related, alternative, and/or more specific or limiting descriptors such as, but not limited to, braided, plaited, interwoven, interweaved, woven, weaved, interlaced, and knitted, whereby each of these terms may equivalently, relatedly, alternatively, or more specifically, be used as an appropriate descriptor for further describing and clarifying the geometrical configuration or construction of expansible, tubular shaped porous mesh-like element 22 of implantable blood filtering device 20, and alternative embodiments thereof, of the present invention.

Preferably, expansible, tubular shaped porous mesh-like element 22, and alternative embodiments thereof, are braided, however, as just described, expansible, tubular shaped porous mesh-like element 22, and alternative embodiments thereof, are each of directly related, alternative, and/or more specific or limiting geometrical configuration or construction, selected from the group consisting of plaited, interwoven, interweaved, woven, weaved, interlaced, and knitted.

Expansible, tubular shaped porous mesh-like element 22 has a first end region e₁ positional in a first source vein (68, FIG. 3A; 62, FIG. 3B; or, 62, FIG. 3C) of the venous furcation (60, FIGS. 3A-3C), a second end region e₂ positional in a second source vein (68, FIG. 3C) or in the sink vein (64, FIGS. 3A and 3B) of the venous furcation (60, FIGS. 3A-3C), and a middle filtering zone F circumferentially and longitudinally extending between first end region e₁ and second end region e₂, whereby middle filtering zone F of mesh-like element 22 when so positioned in the venous furcation, filters the embolic material (solid circles in FIGS. 3A-3C) from the blood passing through openings or pores 26 of middle filtering zone F, while substantially not disturbing flow of the blood through the venous furcation, thereby preventing the embolic material from entering the sink vein of the venous furcation in the subject.

Middle filtering zone F of mesh-like element 22 in the implanted expanded state, refers to a variably geometrically configurable middle region or zone, that is, a continuous segment, circumferentially and longitudinally extending along the middle portion of a longitudinal axis (for example, in FIG. 2A, longitudinal axis 44) of mesh-like element 22 in the implanted expanded state, between first end region e₁ and second end region e₂, of a plurality of adjacent mesh-like element filaments 24, which performs the function of filtering, by way of trapping or capturing, embolic material from the blood flowing from the at least one source vein into the sink vein of a venous furcation, and passing through openings or pores 26 of middle filtering zone F, while substantially not disturbing flow of the blood through the venous furcation, thereby preventing the embolic material from entering the sink vein of the venous furcation and from migrating downstream therefrom in the circulatory system of a subject.

When mesh-like element 22 of blood filtering device 20 is in the implanted expanded deployed state, as indicated in FIGS. 3A-3C, end regions e₁ and e₂ function for anchoring mesh-like element 22 to inner wall regions of the venous furcation, according to actual placement and deployment of mesh-like element 22 inside the venous furcation. Moreover, in addition to first and second end regions e₁ and e₂, essentially all remaining regions of mesh-like element 22 located between middle filtering zone F and first and second end regions e₁ and e₂, also function for anchoring mesh-like element 22 to inner wall regions of the venous furcation. Accordingly, mesh-like element 22 is self-anchoring. Such anchoring enables growth of cells from the vascular inner walls onto surfaces of mesh-like element filaments 24 of mesh-like element 22, so as to incorporate blood filtering device 20 therewith and to prevent pathological damage to the vascular walls due to undesirable accidental movement, displacement, or migration, of the entire, or a portion of, mesh-like element 22.

As stated above, main aspects of novelty and inventiveness of the present invention, are that implantable blood filtering device 20 is designed and constructed specifically for optimally filtering the embolic material from the blood passing through pores of the middle filtering zone, and maintaining a deployed implanted expanded position in the venous furcation, while substantially not disturbing flow of the blood through the venous furcation, thereby highly effectively preventing the embolic material from entering the sink vein of the venous furcation and from migrating downstream therefrom in the circulatory system of the subject.

This is accomplished by geometrically constructing or configuring expansible, tubular shaped porous mesh-like element 22 of blood filtering device 20 according to the above mentioned two types of structural profiles of (1) an inter-region structural profile and (2) intra-region structural profiles, determined by a unique combination of critical ranges of values of dimensional characteristics in the implanted expanded state, according to desired and/or required placement, configuration, and operation of mesh-like element 22 inside the venous furcation.

According to actual requirements of implementation, in specific forms of the preferred embodiment of blood filtering device 20 of the present invention, the geometrical configuration or construction of mesh-like element 22, in the implanted expanded state, is characterized by (1) an inter-region structural profile, whereby values of at least one of the following dimensional characteristics (i)-(viii) from region to region of at least two of the three regions being first end region e₁, second end region e₂, and middle filtering zone F, are either constant or vary, that is, are the same or different, and, characterized by (2) intra-region structural profiles, whereby values of at least one of the following dimensional characteristics (i)-(viii) within one or both of first and second end regions e₁ and e₂, and/or, within middle filtering zone F, are either constant or vary as a function of longitudinal length within each corresponding region along longitudinal axis 44 of mesh-like element 22 in the implanted expanded state. Regarding (2) intra-region structural profiles, the variation is either a continuous variation, or, a non-continuous or discrete variation as a function of longitudinal length within the corresponding region along longitudinal axis 44 of mesh-like element 22 in the implanted expanded state.

This aspect of the inter-region and intra-region structural profiles of the geometrical configuration or construction of mesh-like element 22 directly relates to functional variability and optimization of blood filtering device 20, in general, and to functional variability and optimization of middle filtering zone F and first and second end regions e₁ and e₂, of mesh-like element 22, in particular, as illustratively described in specific examples below.

Dimensional characteristics of blood filtering device 20, in general, and of mesh-like element 22 including middle filtering zone F and first and second end regions e₁ and e₂, in particular, in the implanted expanded state, having critical ranges of values, are illustrated in FIG. 2A, and, in FIG. 2B, a schematic diagram illustrating an enlarged view of a small portion 28 of the first exemplary preferred embodiment of blood filtering device 20 of FIG. 2A. It is herein noted that the following described dimensional characteristics (i)-(viii) are for mesh-like element 22 ‘in the implanted expanded state’, that is, for mesh-like element 22 in the expanded state positioned, implanted, and deployed inside the venous furcation of the subject. These dimensional characteristics and preferred critical ranges of values thereof, of mesh-like element 22 are as follows:

(i) Cross section perimeter, π, of each of mesh-like element filaments 24, has a value in a range of between about 80 μm to about 2500 μm, and preferably, in a range of between about 180 μm to about 1300 μm.

The geometrical shape or form of the cross section of mesh-like element filaments 24 is preferably circular or round, but, in a non-limiting manner, may also be elliptical, square, or rectangular. For example, based on these ranges of values of cross section perimeter, π, of mesh-like element filaments 24, corresponding ranges of values of the diameter of circular or round geometrically shaped or formed mesh-like element filaments 24 are between about 25 μm to about 800 μm, and preferably, between about 60 μm to about 400 μm, respectively.

(ii) Length, W, of a side 30, of an opening or pore 26 formed between mesh-like element filaments 24 in the implanted expanded state, has a value in a range of between about 0.3 mm to about 7 mm, and preferably, in a range of between about 2 mm to about 3 mm.

For example, the length, W, of an exemplary side 32 between points 34 and 36 of exemplary opening or pore 38 (FIG. 2A) formed between mesh-like element filaments 24 in the implanted expanded state.

(iii) Number of mesh-like element filaments 24 of mesh-like element 22, has a value in a range of between about 6 filaments to about 92 filaments, and preferably in a range of between about 18 filaments to about 48 filaments.

For a given dimensional characteristic (viii), luminal length, L, of mesh-like element 22 in the implanted expanded state, the actual number of mesh-like element filaments 24 is indirectly proportional to dimensional characteristic (ii), that is, length, W, of a side 30, of opening or pore 26 formed between mesh-like element filaments 24 in the implanted expanded state. More specifically, for a given luminal length, L, of mesh-like element 22 in the implanted expanded state, the length, W, of a side 30, of opening or pore 26 formed between mesh-like element filaments 24 in the implanted expanded state, increases with decreasing number of mesh-like element filaments 24 of mesh-like element 22.

(iv) Angle, α, of crossed or overlapped mesh-like element filaments 24 in the implanted expanded state, referring to either the right angle, 90°, between two adjacent sides 40 and 42 of a square shaped opening or pore 26, formed between crossed or overlapped mesh-like element filaments 24 in the implanted expanded state, or, referring to the obtuse angle, between 90° and 180°, between two adjacent sides 40 and 42 of a non-square, parallelogram, shaped, opening or pore 26, formed between crossed or overlapped mesh-like element filaments 24 in the implanted expanded state, having a value in a range of between about 95° to about 140°, and preferably, in a range of between about 110° to about 120°.

For example, FIG. 2B illustrates the later case, wherein the angle, α, of crossed or overlapped mesh-like element filaments 24 in the implanted expanded state, refers to the obtuse angle, between 90° and 180°, between adjacent sides 40 and 42 of non-square, parallelogram, shaped, opening or pore 26, formed between crossed or overlapped mesh-like element filaments 24 in the implanted expanded state.

(v) Pitch, P, of turnings of mesh-like element filaments 24 in the implanted expanded state, referring to the distance, along a same longitudinal axis of mesh-like element 22, between two corresponding points located on adjacent turnings of mesh-like element filaments 24 in the implanted expanded state, has a value in a range of between about 0.5 mm to about 10 mm, and preferably, in a range of between about 2.5 mm to about 4 mm.

For example, as shown in FIG. 2A, pitch, P, of turnings of mesh-like element filaments 24 in the implanted expanded state, refers to the distance along longitudinal axis 44 of mesh-like element 22, between corresponding points 46 and 48 located on adjacent turnings of mesh-like element filaments 24 in the implanted expanded state. In another view, as shown in FIG. 2B, pitch, P, of turnings of mesh-like element filaments 24 in the implanted expanded state, refers to the distance along longitudinal axis (the dashed horizontal line) 50 of mesh-like element 22, between corresponding points 52 and 54 located on adjacent turnings of mesh-like element filaments 24 in the implanted expanded state.

In general, decreasing pitch, P, of turnings of mesh-like element filaments 24 in the implanted expanded state, of a particular region or regions, for example, of first end region e₁, and/or of second end region e₂, and/or of middle filtering zone F, increases the radial force generated by filaments 24 of the particular region or regions upon the inner wall regions at the respective position or positions inside the venous furcation. This phenomenon is especially exploited by selecting a particular pitch, P, of turnings of mesh-like element filaments 24 in the implanted expanded state, of one or both of first and second end regions e₁ and e₂, having a value less than that of middle filtering zone F, where first and second end regions e₁ and e₂ primarily function for anchoring mesh-like element 22 to inner wall regions of the venous furcation, according to actual placement and deployment of mesh-like element 22 inside the venous furcation.

(vi) Porosity index of mesh-like element 22 in the implanted expanded state, has a value in a range of between about 50% to about 95%, and preferably, in a range of between about 70% to about 85%.

Herein, the porosity index of mesh-like element 22 is defined as the ratio of the total ‘empty’ circumferentially and longitudinally extending area of all openings or pores 26 formed between mesh-like element filaments 24 to the total ‘empty’ plus ‘occupied’ circumferentially and longitudinally extending area of mesh-like element 22, in the implanted expanded state. The total circumferentially and longitudinally extending area of mesh-like element 22 corresponds to the sum of the total ‘empty’ circumferentially and longitudinally extending area of all openings or pores 26 formed between mesh-like element filaments 24 and the total ‘occupied’ circumferentially and longitudinally extending area of all mesh-like element filaments 24, in the implanted expanded state.

For a given value of dimensional characteristic (i), that is, cross section perimeter, π, of mesh-like element filaments 24, the porosity index of mesh-like element 22 in the implanted expanded state is directly proportional to dimensional characteristic (v), that is, pitch, P, of turnings of mesh-like element filaments 24 in the implanted expanded state. More specifically, the larger is pitch, P, of turnings of mesh-like element filaments 24 in the implanted expanded state, the larger is the porosity index of mesh-like element 22 in the implanted expanded state.

(vii) Diameter, D, of mesh-like element 22 in the implanted expanded state, has a value in a range of between about 5 mm to about 40 mm.

For application to a femoral vein type of venous bifurcation, diameter, D, of mesh-like element 22 in the implanted expanded state, has a value in a range of between about 5 mm to about 25 mm, and preferably, in a range of between about 10 mm to about 20 mm. For application to an inferior vena cava vein type of venous bifurcation, diameter, D, of mesh-like element 22 in the implanted expanded state, has a value in a range of between about 10 mm to about 40 mm, and preferably, in a range of between about 15 mm to about 30 mm.

As a first example, for application to an iliac vein-inferior vena cava vein type of venous bifurcation, diameter, D, of mesh-like element 22 in the implanted expanded state, has a value in a range of between about 10 mm to about 40 mm, and preferably, in a range of between about 15 mm to about 30 mm. As a second example, for application to an iliac vein-iliac vein type of venous bifurcation, diameter, D, of mesh-like element 22 in the implanted expanded state, has a value in a range of between about 5 mm to about 15 mm, and preferably, in a range of between about 10 mm to about 15 mm.

For introduction into the vascular system of a subject, mesh-like element 22 is radially compressed, whereby diameter, D, of mesh-like element 22 in the contracted state, has a value in a range of between about 1.3 mm to about 1.7 mm.

(viii) Luminal length, L, of mesh-like element 22 in the implanted expanded state, has a value in a range of between about 15 mm to about 200 mm, and preferably, in a range of between about 30 mm to about 100 mm. The actual luminal length, L, in the implanted expanded state, varies according to the intended use and anatomical position of mesh-like element 22 at the venous furcation.

For introduction into the vascular system of a subject, mesh-like element 22 is radially compressed and elongates, whereby luminal length, L, of mesh-like element 22 in the contracted state, is longer than that in the implanted expanded state by an amount in a range of between about 50% to about 500%. Accordingly, based on the previously indicated ranges of luminal length, L, of mesh-like element 22 in the implanted expanded state, the luminal length of mesh-like element 22 in the contracted state, has a value in a range of between about 22 mm to about 1000 mm.

Combination of the above described preferred critical ranges of values of the dimensional characteristics (i) through (viii) is substantially different from combinations of ranges of values of the same or similar dimensional characteristics of prior art intravascular or intraluminal blood filtering devices, and, of other prior art intravascular or intraluminal tubular mesh-like porous devices, such as braided stents.

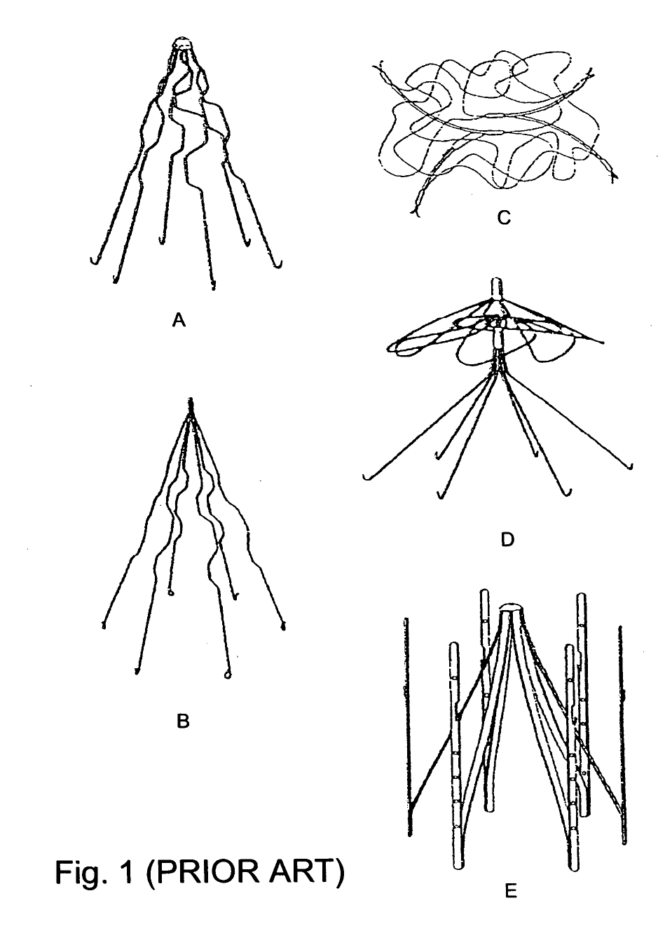

For example, focusing on characteristic dimension (i), the cross section perimeter, π, the preferred range of values of the diameter of circular or round geometrically shaped or formed mesh-like element filaments 24, of blood filtering device 20 of the present invention, is between about 60 μm to about 400 μm. By strong contrast, the Kimmell or Greenfield blood clot filter, as disclosed in above cited U.S. Pat. No. 3,952,747, being an example of the first general type of vena cava filter previously described above and illustrated in FIG. 1 (A), features filaments having a typical diameter of about 450 μm. Additionally, the bird's nest type blood filter, as disclosed in above cited U.S. Pat. No. 4,494,531, being an example of the second type of vena cava filter previously described above and illustrated in FIG. 1 (C), features filaments having a typical diameter of about 180 μm.

In each of these prior art blood filtering devices, which are just two specific examples of blood filtering devices currently used for treating and/or preventing conditions associated with embolic material in blood flowing in the vicinity of the vena cava, the filaments are significantly wider than mesh-like element filaments 24 of blood filtering device 20 of the present invention, which has important implications regarding intravascular performance, and potential undesirable side effects caused by the presence, of a blood filtering device in the vascular system of a subject. In particular, with regard to thrombogenicity, deep venous thrombosis (DVT), and unfavorable change to the hemodynamics flow profile caused by the presence of relatively large diameter filaments of a blood filtering device, as a direct result of mesh-like element filaments 24 of blood filtering device 20 of the present invention being sufficiently thin so as to negligibly alter the flow of blood through a venous furcation, in strong contrast to filaments of prior art intravascular or intraluminal blood filtering devices, local production of thrombi or other embolic material, occurrence of DVT, unfavorable change to the hemodynamics flow profile, and/or reduction in filter patency, at or in the vicinity of the emplacement site are essentially eliminated.

As stated above, according to actual requirements of implementation, in specific forms of the preferred embodiment of blood filtering device 20 of the present invention, the geometrical configuration or construction of mesh-like element 22 is characterized by (1) an inter-region structural profile, whereby values of at least one of the dimensional characteristics (i)-(viii) from region to region of at least two of the three regions being first end region e₁, second end region e₂, and, middle filtering zone F, are either constant or vary, that is, are the same or different, and, characterized by (2) intra-region structural profiles, whereby, whereby values of at least one of the dimensional characteristics (i)-(viii) within one or both of first and second end regions e₁ and e₂, and/or, within middle filtering zone F, are either constant or vary as a function of longitudinal length within each corresponding region along longitudinal axis 44 of mesh-like element 22 in the implanted expanded state.

Accordingly, the inter-region structural profile of mesh-like element 22 corresponds to the comparison that is, the sameness or difference, of the specific geometrical configuration or construction from region to region of at least two regions selected from the three regions being first and second end regions e₁ and e₂, and middle filtering zone F, of mesh-like element 22. Specifically, the comparison, that is, the sameness or difference, from region to region, of values in the set of values of above described dimensional characteristics (i) through (viii) of each of first and second end regions e₁ and e₂, and, corresponding values in the set of values of dimensional characteristics (i) through (viii) of middle filtering zone F, of mesh-like element 22.

More specifically, the comparison, that is, the sameness or difference, from region to region of values of dimensional characteristics of (i) cross section perimeter, π, of mesh-like element filaments 24, of each of first and second end regions e₁ and e₂, and, of middle filtering zone F, (ii) length, W, of a side of the opening or pore 26 formed between mesh-like element filaments 24 in the implanted expanded state, of each of first and second end regions e₁ and e₂, and, of middle filtering zone F, (iii) number of mesh-like element filaments 24, of each of first and second end regions e₁ and e₂, and, of middle filtering zone F, (iv) angle, α, of crossed or overlapped mesh-like element filaments 24 in the implanted expanded state, referring to the obtuse angle, between 90° and 180°, between adjacent sides of the non-square, parallelogram, shaped, opening or pore formed between crossed or overlapped mesh-like element filaments 24 in the implanted expanded state, of each of first and second end regions e₁ and e₂, and, of middle filtering zone F, (v) pitch, P, of turnings of mesh-like element filaments 24 in the implanted expanded state, of each of first and second end regions e₁ and e₂, and, of middle filtering zone F, (vi) porosity index in the implanted expanded state, of each of first and second end regions e₁ and e₂, and, of middle filtering zone F, (vii) diameter, D, in the implanted expanded state, of each of first and second end regions e₁ and e₂, and, of middle filtering zone F, and, (viii) luminal length, L, in the implanted expanded state, of each of first and second end regions e₁ and e₂, that is, L₁ and L₂, respectively, and, of middle filtering zone F, that is, L_{F}, of mesh-like element 22.

Additionally, the intra-region structural profiles correspond to the constancy or variability of values of dimensional characteristics (i)-(viii) within one or both of first and second end regions e₁ and e₂, and/or, within middle filtering zone F, as a function of longitudinal length within each corresponding region along longitudinal axis 44 of mesh-like element 22 in the implanted expanded state.

With respect to (1) the inter-region structural profile of the geometrical configuration or construction of mesh-like element 22, as shown in FIG. 2A, as an illustrative example, values in the set of values of dimensional characteristics (i) through (viii) of each of first and second end regions e₁ and e₂, are shown as being the same as corresponding values in the set of values of dimensional characteristics (i) through (viii) of middle filtering zone F, of mesh-like element 22. Accordingly, values in the set of values of dimensional characteristics (i) through (viii) of first end region e₁ are shown as being the same as corresponding values in the set of values of dimensional characteristics (i) through (viii) of second end region e₂, of mesh-like element 22.

Moreover, with respect to (2) intra-region structural profiles, as shown in FIG. 2A, values of dimensional characteristics (i)-(viii) within each of first and second end regions e₁ and e₂, and, within middle filtering zone F, are constant as a function of longitudinal length within each corresponding region along longitudinal axis 44 of mesh-like element 22 in the implanted expanded state.

Thus, the overall structural profile of mesh-like element 22 of blood filtering device 20, as illustrated in FIG. 2A, is characterized by the same set of constant values of dimensional characteristics (i) through (viii) from region to region among and within all three regions being first and second end regions e₁ and e₂, and, middle filtering zone F, of mesh-like element 22.

FIGS. 3A-3C are schematic diagrams illustrating three alternative types of deployment of the exemplary preferred embodiment of the implantable blood filtering device of FIG. 2A, that is, blood filtering device 20, in a venous furcation in a subject, where for illustrative purposes, the venous furcation is a venous bifurcation 60. In each of these illustrations, an arrow shows a known or anticipated direction of travel of embolic material (indicated by solid circles) in the blood flowing from at least one of source veins 62 and 68 towards and into sink vein 64 of venous bifurcation 60. The known or anticipated direction of travel of the embolic material in the flowing blood is used to determine where most effectively to implant and deploy blood filtering device 20, according to a particular clinical situation.

In FIG. 3A, implantable blood filtering device 20 is implanted and deployed in venous bifurcation 60 in a subject, whereby middle filtering zone F of expansible, tubular shaped porous mesh-like element 22 in the implanted expanded state filters embolic material (solid circles) from blood flowing from one source vein 62 towards and into sink vein 64 of venous bifurcation 60, thereby preventing the embolic material from entering sink vein 64 of venous bifurcation 60 and from migrating further downstream 66 therefrom in the circulatory system of the subject.

As shown in FIG. 3A, mesh-like element 22 has first end region e₁ positional in a first source vein 68 of venous furcation 60, second end region e₂ positional in the sink vein 64 of venous furcation 60, and middle filtering zone F circumferentially and longitudinally extending between first end region e₁ and second end region e₂, whereby middle filtering zone F of mesh-like element 22 when so positioned in venous furcation 60, filters the embolic material (solid circles) from the blood passing through pores 26 of middle filtering zone F, while substantially not disturbing flow of the blood through venous furcation 60, thereby preventing the embolic material from entering sink vein 64 of venous furcation 60 in the subject, and from migrating further downstream 76 therefrom in the circulatory system of the subject.

In FIG. 3B, implantable blood filtering device 20 is implanted and deployed in venous bifurcation 60 in a subject, whereby middle filtering zone F of expansible, tubular shaped porous mesh-like element 22 in the implanted expanded state filters embolic material (solid circles) from blood flowing from one source vein 68 towards and into sink vein 64 of venous bifurcation 60, thereby preventing the embolic material from entering sink vein 64 of venous bifurcation 60 and from migrating further downstream 66 therefrom in the circulatory system of the subject.

As shown in FIG. 3B, mesh-like element 22 has first end region e₁ positional in a first source vein 62 of venous furcation 60, second end region e₂ positional in the sink vein 64 of venous furcation 60, and middle filtering zone F circumferentially and longitudinally extending between first end region e₁ and second end region e₂, whereby middle filtering zone F of mesh-like element 22 when so positioned in venous furcation 60, filters the embolic material (solid circles) from the blood passing through pores 26 of middle filtering zone F, while substantially not disturbing flow of the blood through venous furcation 60, thereby preventing the embolic material from entering sink vein 64 of venous furcation 60 in the subject, and from migrating further downstream 76 therefrom in the circulatory system of the subject.

In FIG. 3C, implantable blood filtering device 20 is implanted and deployed in venous bifurcation 60 in a subject, whereby middle filtering zone F of expansible, tubular shaped porous mesh-like element 22 in the implanted expanded state filters embolic material (solid circles) from blood flowing from both source veins 62 and 68 towards and into sink vein 64 of venous bifurcation 60, thereby preventing the embolic material from entering sink vein 64 of venous bifurcation 60 and from migrating further downstream 66 therefrom in the circulatory system of the subject.

As shown in FIG. 3C, mesh-like element 22 has first end region e₁ positional in first source vein 62 of venous furcation 60, second end region e₂ positional in second source vein 68 of venous furcation 60, and middle filtering zone F circumferentially and longitudinally extending between first end region e₁ and second end region e₂, whereby middle filtering zone F of mesh-like element 22 when so positioned in venous furcation 60, filters the embolic material (solid circles) from the blood passing through pores 26 of middle filtering zone F, while substantially not disturbing flow of the blood through venous furcation 60, thereby preventing the embolic material from entering sink vein 64 of venous furcation 60 in the subject, and from migrating further downstream 76 therefrom in the circulatory system of the subject.

As shown in FIGS. 3A-3C, first and second end regions e₁ and e₂, of mesh-like element 22 in the implanted expanded deployed state function for anchoring mesh-like element 22 to inner wall regions of venous bifurcation 60. Moreover, in addition to first and second end regions e₁ and e₂, essentially all remaining regions of mesh-like element 22 located between variable middle filtering zone F and first and second end regions e₁ and e₂, also function for anchoring mesh-like element 22 to inner wall regions of venous bifurcation 60. As previously stated, such anchoring enables growth of cells from the vascular inner walls onto surfaces of mesh-like element filaments 24 of mesh-like element 22, so as to incorporate blood filtering device 20 therewith and to prevent pathological damage to the vascular walls due to undesirable accidental movement, displacement, or migration, of the entire, or a portion of, mesh-like element 22.

A first illustrative and descriptive example of exploiting the aspect of variable geometrical configuration or construction, in general, with respect to variable inter-region structural profile, in particular, of mesh-like element 22 of blood filtering device 20, is provided herein as follows. The objective here is for providing an alternative embodiment of blood filtering device 20 which optimally filters the embolic material from the blood passing through pores of the middle filtering zone, and maintaining a deployed implanted expanded position in the venous furcation, while substantially not disturbing flow of the blood through the venous furcation.

Positioning and deployment of blood filtering device 20, of FIG. 2A, featuring mesh-like element 22, including middle filtering zone F and first and second end regions e₁ and e₂, having the same sets of constant values of dimensional characteristics (i) through (viii), in a venous furcation, for example, in venous bifurcation 60, according to the above described alternative types of positioning and deployment illustrated in FIGS. 3A-3C, in general, and especially according to the above described third type of positioning and deployment illustrated in FIG. 3C, in particular, may result in improper or insufficient anchoring of first end region e₁ to the inner wall region of first source vein 62, and/or, improper or insufficient anchoring of second end region e₂ to the inner wall region of second source vein 68, in particular, and improper or insufficient anchoring of mesh-like element 22 to the inner wall regions of venous bifurcation 60, in general, thereby potentially leading to pathological damage to the vascular walls due to undesirable accidental movement, displacement, or migration, of a portion of, or the entire, mesh-like element 22.

The above described undesirable potential situation is prevented by geometrically constructing or configuring mesh-like element 22 of blood filtering device 20 according to specific inter-region and/or intra-region structural profiles, determined by a unique combination of critical ranges of values of selected, that is, one or more, above described dimensional characteristics (i) through (viii), which, with reference to FIGS. 3A-3C, in general, and FIG. 3C, in particular, enables proper and sufficient anchoring of first end region e₁ to the inner wall region of first source vein 62 and proper and sufficient anchoring of second end region e₂ to the inner wall region of second source vein 68, in particular, and proper and sufficient anchoring of mesh-like element 22 to the inner wall regions of venous bifurcation 60, in general. Thus, fulfilling the above stated objective of providing an alternative embodiment of blood filtering device 20 which optimally filters the embolic material from the blood passing through pores 26 of middle filtering zone F, and maintaining a deployed implanted expanded position in venous bifurcation 60, while substantially not disturbing flow of the blood through venous bifurcation 60.

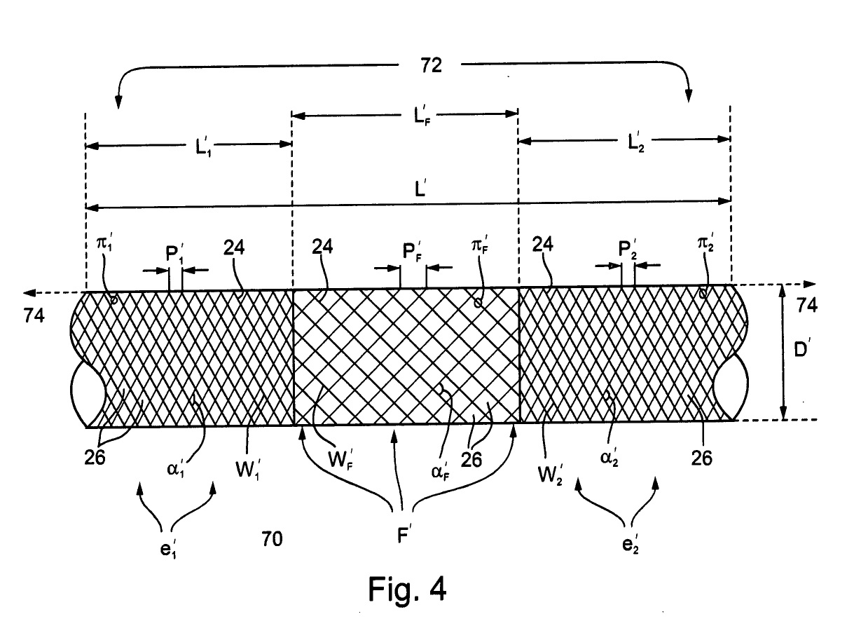

Specifically, mesh-like element 22, including first and second end regions e₁ and e₂, and middle filtering zone F, is geometrically constructed or configured with a variable inter-region structural profile, whereby values of selected dimensional characteristics (i) through (viii) from region to region of first and second end regions e₁ and e₂, are different from values of corresponding selected dimensional characteristics (i) through (viii) of middle filtering zone F, in the implanted expanded state. More specifically, mesh-like element 22 is geometrically constructed or configured with a particular inter-region structural profile, whereby the values of dimensional characteristics (ii), (iii), (iv), (v), and (vi), from region to region of first and second end regions e₁ and e₂, are different from the values of corresponding dimensional characteristics (ii), (iii), (iv), (v), and (vi), of middle filtering zone F, as described immediately below and illustrated in FIG. 4.

FIG. 4 is a schematic diagram illustrating an exemplary preferred embodiment of a first alternative form of implantable blood filtering device 20 of FIGS. 2A and 2B, herein, for brevity, generally referred to as blood filtering device 70, wherein the geometrical configuration or construction is characterized by a variable inter-region structural profile, whereby values of dimensional characteristics (ii)-(vi) of first and second end regions e₁′ and e₂′ are notably different from the corresponding values of dimensional characteristics (ii)-(vi) of middle filtering zone F′.

As for blood filtering device 20, blood filtering device 70 is an expansible, tubular shaped porous mesh-like element 72, herein, also referred to as mesh-like element 72, formed from the previously mentioned mesh-like filaments, fibers, wires, or strands 24, or, for brevity, filaments 24. Mesh-like element 72 has openings or pores 26, formed and located in between adjacent mesh-like filaments 24, circumferentially and longitudinally extending along the entirety of mesh-like element 72.

Similar to that indicated in FIGS. 3A-3C for mesh-like element 22 of blood filtering device 20, mesh-like element 72, shown in FIG. 4, has a first end region e₁′ positional in a first source vein (68, FIG. 3A; 62, FIG. 3B; or, 62, FIG. 3C) of the venous furcation (60, FIGS. 3A-3C), a second end region e₂′ positional in a second source vein (68, FIG. 3C) or in the sink vein (64, FIGS. 3A and 3B) of the venous furcation (60, FIGS. 3A-3C), and a middle filtering zone F′ circumferentially and longitudinally extending between first end region e₁′ and second end region e₂′, whereby middle filtering zone F′ of mesh-like element 72 when so positioned in the venous furcation, filters the embolic material (solid circles) from the blood passing through openings or pores 26 of middle filtering zone F′, while substantially not disturbing flow of the blood through the venous furcation, thereby preventing the embolic material from entering the sink vein of the venous furcation in the subject.

Middle filtering zone F′ of mesh-like element 72 in the implanted expanded state, is a variably geometrically configurable middle zone or region, that is, a continuous segment, circumferentially and longitudinally extending along the middle portion of a longitudinal axis (for example, in FIG. 4, longitudinal axis 74) of mesh-like element 72 in the implanted expanded state, between first end region e₁′ and second end region e₂′, of a plurality of adjacent mesh-like element filaments 24, which performs the function of filtering, by way of trapping or capturing, embolic material from the blood flowing from the at least one source vein into the sink vein of a venous furcation, and passing through openings or pores 26 of middle filtering zone F′, while substantially not disturbing flow of the blood through the venous furcation, thereby preventing the embolic material from entering the sink vein of the venous furcation and from migrating downstream therefrom in the circulatory system of a subject.

When mesh-like element 72 of blood filtering device 70 is in the implanted expanded deployed state, similar to that indicated in FIGS. 3A-3C for mesh-like element 22 of blood filtering device 20, first and second end regions e₁′ and e₂′ function for anchoring mesh-like element 72 to inner wall regions of the venous furcation, according to actual placement and deployment of mesh-like element 72 inside the venous furcation. Moreover, in addition to first and second end regions e₁′ and e₂′, essentially all remaining regions of mesh-like element 72 located between middle filtering zone F′ and first and second end regions e₁′ and e₂′, also function for anchoring mesh-like element 72 to inner wall regions of the venous furcation. Accordingly, mesh-like element 72 is self-anchoring. Such anchoring enables growth of cells from the vascular inner walls onto surfaces of mesh-like element filaments 24 of mesh-like element 72, so as to incorporate blood filtering device 70 therewith and to prevent pathological damage to the vascular walls due to undesirable accidental movement, displacement, or migration, of the entire, or a portion of, mesh-like element 72.

As shown in FIG. 4, with respect to the previously described inter-region structural profile, relating to comparison between specific geometrical configurations or constructions, several values in the set of values of dimensional characteristics (i) through (viii), of first and second end regions e₁′ and e₂′ are shown as being significantly different from corresponding values in the set of values of dimensional characteristics (i) through (viii), of middle filtering zone F′ of mesh-like element 72. More specifically, for illustrative example, first and second end regions e₁′ and e₂′ are shown in FIG. 4 as having different values of dimensional characteristics of (ii), (iii), (iv), (v), and (vi), compared to, that is, greater than or less than, corresponding values of dimensional characteristics (ii), (iii), (iv), (v), and (vi), of middle filtering zone F′, of mesh-like element 72. This corresponds to a variable inter-region structural profile characterizing the geometrical configuration or construction of mesh-like element 72 in the implanted expanded state, as illustratively described in detail immediately following.

With respect to dimensional characteristic (ii), the value of length, W₁′ and W₂′, of a side of the opening or pore 26 formed between mesh-like element filaments 24 in the implanted expanded state, of first and second end regions e₁′ and e₂′, respectively, is less than the corresponding value of length, W_{F}′, of middle filtering zone F′.

With respect to dimensional characteristic (iii), the value of number of mesh-like element filaments 24, of each of first and second end regions e₁′ and e₂′, is greater than the corresponding value of number of mesh-like element filaments 24, of middle filtering zone F′.

With respect to dimensional characteristic (iv), the value of angle, α₁′, and α₂′, the obtuse angle, between 90° and 180°, between adjacent sides of the non-square, parallelogram, shaped, opening or pore 26 formed between crossed or overlapped mesh-like element filaments 24 in the implanted expanded state, of first and second end regions e₁′ and e₂′, respectively, is greater than the corresponding value of angle, α_{F}′, of 90°, between adjacent sides of the square shaped opening or pore 26 formed between crossed or overlapped mesh-like element filaments 24 in the implanted expanded state, of middle filtering zone F′.

With respect to dimensional characteristic (v), the value of pitch, P₁′ and P₂′, of first and second end regions e₁′ and e₂′, respectively, of turnings of mesh-like element filaments 24 in the implanted expanded state, is less than the corresponding value of pitch, P_{F}′, of middle filtering zone F′.

With respect to dimensional characteristic (vi), the value of the porosity index in the implanted expanded state, of each of first and second end regions e₁′ and e₂′, is less than the corresponding value of the porosity index of middle filtering zone F′. As previously described and illustrated above with reference to the porosity index of mesh-like element 22 (FIGS. 2A and 2B), here, with reference to mesh-like element 72, as illustrated in FIG. 4, in the implanted expanded state, for a given value of dimensional characteristic (i), that is, cross section perimeter, π, of mesh-like element filaments 24, the porosity index of each of first and second end regions e₁′ and e₂′, and of middle filtering zone F′, the porosity index is directly proportional to dimensional characteristic (v), that is, pitch, P₁′ and P₂′, of first and second end regions e₁′ and e₂′, respectively, and, pitch, P_{F}′, of middle filtering zone F′.

It is especially noted, that for mesh-like element 72 of blood filtering device 70, the smaller value of pitch, P₁′ and P₂′, and the smaller value of the porosity index, of first and second end regions e₁′ and e₂′, respectively, compared to the corresponding values of these dimensional characteristics of middle filtering zone F′, are so selected whereby, with reference to FIGS. 3A-3C, in general, and FIG. 3C, in particular, there is a greater overall structural and mechanical strength provided by blood filtering device 70, including an increase of anchoring of mesh-like element 72 to inner wall regions of a venous furcation, compared to mesh-like element 22 of blood filtering device 20, according to actual placement and deployment of mesh-like element 72 inside the venous furcation. This is a direct result of the previously described phenomenon whereby, in general, decreasing pitch, P, of turnings of mesh-like element filaments 24 in the implanted expanded state, of a particular region or regions, for example, in this case, of first and second end regions e₁ and e₂, increases the radial force generated by the particular region or regions, that is, first and second end regions e₁ and e₂, upon the inner wall regions at the respective position or positions of first and second end regions e₁ and e₂ inside the venous furcation.

Simultaneously, the smaller value of pitch, P₁′ and P₂′, and the smaller value of the porosity index, of first and second end regions e₁′ and e₂′, respectively, compared to the corresponding values of these dimensional characteristics of middle filtering zone F′, of mesh-like element 72 of blood filtering device 70, are selected so as to advantageously fulfill the above stated objective of providing an alternative embodiment of the blood filtering device of the present invention which optimally filters the embolic material from the blood passing through pores of the middle filtering zone, and maintaining a deployed implanted expanded position in a venous bifurcation, while substantially not disturbing flow of the blood through the venous bifurcation.

For the purpose of completeness of description and illustration of the present invention, in a non-limiting manner, each of first and second end regions e₁′ and e₂′ is shown in FIG. 4 as having the same values of dimensional characteristics of (i), (vii), and (viii), compared to corresponding values of dimensional characteristics (i), (vii), and (viii), of middle filtering zone F′, of mesh-like element 72, as described immediately following.

With respect to dimensional characteristic (i), the value of cross section perimeter, π₁′ and π₂′, of mesh-like element filaments 24, of first and second end regions e₁′ and e₂′, respectively, is the same as the corresponding value of cross section perimeter, π_{F}′, of mesh-like element filaments 24, of middle filtering zone F′. With respect to dimensional characteristic (vii), the value of diameter, D′, of each of first and second end regions e₁′ and e₂′, respectively, is the same as the corresponding value of diameter, D′, of middle filtering zone F′, of mesh-like element 72 in the implanted expanded state. With respect to dimensional characteristic (viii), the value of luminal length, L₁′ and L₂′, of first and second end regions e₁′ and e₂′, respectively, is the same as the corresponding value of luminal length, L_{F}′, of variable middle filtering zone F′, of mesh-like element 72 in the implanted expanded state.

Moreover, with respect to the intra-region structural profiles characterizing the geometrical configuration or construction of mesh-like element 72 of blood filtering device 70, as shown in FIG. 4, the entire set of values of dimensional characteristics (i)-(viii) within each of first and second end regions e₁′ and e₂′, and within middle filtering zone F′, are constant as a function of longitudinal length within each corresponding region along longitudinal axis 74 of mesh-like element 72 in the implanted expanded state.

A second illustrative and descriptive example of exploiting the aspect of variable geometrical configuration or construction, in general, with respect to variable inter-region structural profile, in particular, of mesh-like element 22 of blood filtering device 20, is provided herein as follows. As for the preceding example, the objective here is for providing an alternative embodiment of blood filtering device 20 which optimally filters the embolic material from the blood passing through pores of the middle filtering zone, and maintaining a deployed implanted expanded position in the venous furcation, while substantially not disturbing flow of the blood through the venous furcation.

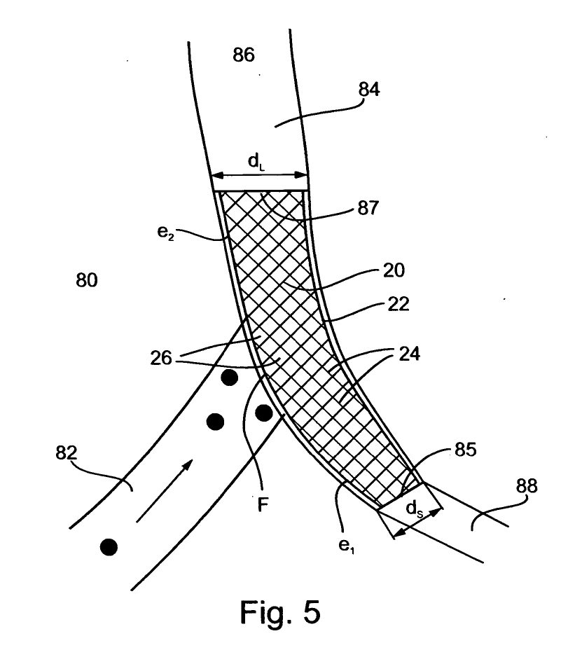

Positioning and deployment of blood filtering device 20, of FIG. 2A, featuring mesh-like element 22, including middle filtering zone F and first and second end regions e₁ and e₂, having the same sets of constant values of dimensional characteristics (i) through (viii), in a venous furcation, for example, in venous bifurcation 60, according to the above described first and second alternative types of positioning and deployment illustrated in FIGS. 3A and 3B, respectively, where, for example, the diameter of the source vein (68, FIG. 3A; 62, FIG. 3B, respectively) at the extremity of first end region e₁ is smaller than the diameter of the sink vein 64 at the extremity of second end region e₂ (such as that schematically illustrated in FIG. 5 and described immediately following), may result in improper or insufficient anchoring of second end region e₂ to the larger diameter inner wall region of sink vein 64, in particular, and improper or insufficient anchoring of mesh-like element 22 to the inner wall regions of venous bifurcation 60, in general, thereby potentially leading to pathological damage to the vascular walls due to undesirable accidental movement, displacement, or migration, of a portion of, or the entire, mesh-like element 22.

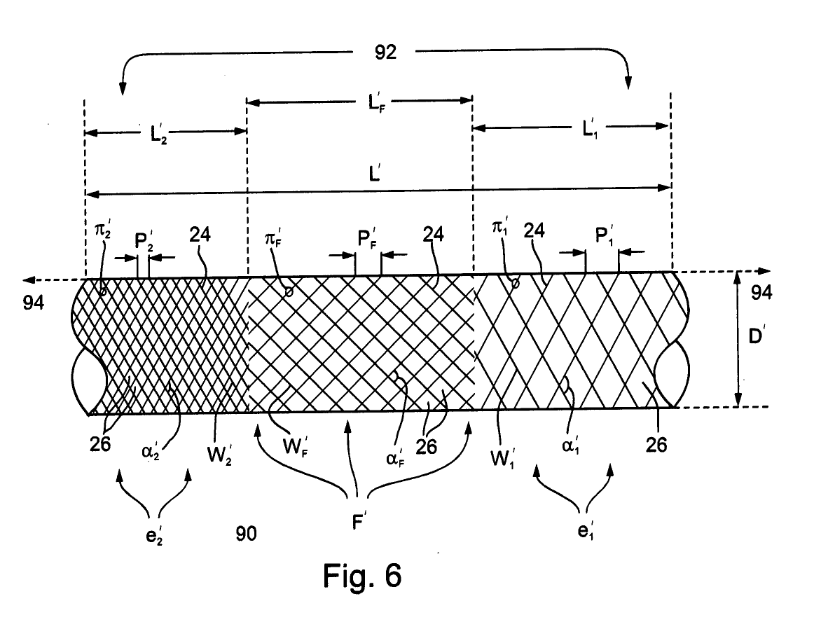

FIG. 5 is a schematic diagram more specifically illustrating the above described structural/functional blood filtering device implementation problem commonly existing in blood vessels which are part of a venous furcation, which is overcome by using the second alternative form of implantable blood filtering device 20 of FIGS. 2A-2B, illustrated in FIG. 6. In FIG. 5, the embodiment of implantable blood filtering device 20 as shown in FIG. 2A, featuring mesh-like element 22, including middle filtering zone F and first and second end regions e₁ and e₂, having the same set of constant values of dimensional characteristics (i) through (viii), is to be implanted and deployed in venous bifurcation 80 in a subject, whereby middle filtering zone F of expansible, tubular shaped porous mesh-like element 22 in the implanted expanded state filters embolic material (solid circles) from blood flowing from one source vein 82 towards and into sink vein 84 of venous bifurcation 80, thereby preventing the embolic material from entering sink vein 84 of venous bifurcation 80 and from migrating further downstream 86 therefrom in the circulatory system of the subject.

As shown in FIG. 5, mesh-like element 22 has first end region e₁ positional in a first source vein 88 of venous bifurcation 80, second end region e₂ positional in the sink vein 84 of venous bifurcation 80, and middle filtering zone F circumferentially and longitudinally extending between first end region e₁ and second end region e₂, whereby middle filtering zone F of mesh-like element 22 when so positioned in venous bifurcation 80, filters the embolic material from the blood passing through pores 26 of middle filtering zone F, while substantially not disturbing flow of the blood through venous bifurcation 80. In the type of deployment shown in FIG. 5, the diameter, d_{S}, of source vein 88 at extremity 85 of first end region e₁ is smaller than the diameter, d_{L}, of sink vein 84 at extremity 87 of second end region e₂.

If blood filtering device 20, including middle filtering zone F and first and second end regions e₁ and e₂, of mesh-like element 22 in the implanted expanded state, featuring the same constant diameter, D, is implanted and deployed in such a variable diameter venous bifurcation, without inter-region variation of angle, α, of crossed or overlapped mesh-like element filaments 24 in the implanted expanded state, and/or pitch, P, of turnings of mesh-like element filaments 24 in the implanted expanded state, along a longitudinal axis of mesh-like element 22, may result in improper or insufficient anchoring of second end region e₂ to the larger diameter inner wall region of sink vein 84, in particular, and improper or insufficient anchoring of mesh-like element 22 to the inner wall regions of venous bifurcation 80, in general, thereby potentially leading to pathological damage to the vascular walls due to undesirable accidental movement, displacement, or migration, of a portion of, or the entire, mesh-like element 22.

The above described undesirable potential situation is prevented by geometrically constructing or configuring mesh-like element 22 of blood filtering device 20 according to a specific inter-region structural profile, determined by a unique combination of critical ranges of values of selected, that is, one or more, above described dimensional characteristics (i) through (viii), which, with reference to FIG. 5, enables proper and sufficient anchoring of second end region e₂ to the larger diameter inner wall region of sink vein 84, in particular, and proper and sufficient anchoring of mesh-like element 22 to the inner wall regions of venous bifurcation 80, in general. Thus, fulfilling the previously stated objective of providing an alternative embodiment of blood filtering device 20 which optimally filters the embolic material from the blood passing through pores 26 of middle filtering zone F, and maintaining a deployed implanted expanded position in variable diameter venous bifurcation 80, while substantially not disturbing flow of the blood through venous bifurcation 80.

Specifically, mesh-like element 22, including variable middle filtering zone F and first and second end regions e₁ and e₂, is geometrically constructed or configured with a variable inter-region structural profile, whereby values of selected dimensional characteristics (i)-(viii) from region to region of middle filtering zone F and both first and second end regions e₁ and e₂, in the implanted expanded state, vary, that is, are notably different. More specifically, mesh-like element 22 is geometrically constructed or configured with a particular inter-region structural profile, whereby values of dimensional characteristics (ii), (iii), (iv), (v), and (vi), from region to region of each of the three regions, that is, first end region e₁, second end region e₂, and middle filtering zone F, are notably different, as described immediately below and illustrated in FIG. 6.

FIG. 6 is a schematic diagram illustrating an exemplary preferred embodiment of a second alternative form of implantable blood filtering device 20 of FIGS. 2A and 2B, herein, for brevity, generally referred to as blood filtering device 90, wherein the geometrical configuration or construction is characterized by a variable inter-region structural profile, whereby values of dimensional characteristics (ii)-(vi) from region to region of each of the three regions, that is, first end region e₁′, second end region e₂′, and middle filtering zone F′, are notably different. As for blood filtering device 20, previously described above and illustrated in FIGS. 2A-2B, blood filtering device 90 is an expansible, tubular shaped porous mesh-like element 92, herein, also referred to as mesh-like element 92, formed from mesh-like filaments, fibers, wires, or strands 24. Mesh-like element 92 has openings or pores 26, formed and located in between adjacent mesh-like filaments 24, circumferentially and longitudinally extending along the entirety of mesh-like element 92.

Similar to that previously described above regarding mesh-like element 22 of blood filtering device 20, as illustrated in FIGS. 3A-3C, and, regarding mesh-like element 72, as illustrated in FIG. 4, here, mesh-like element 92 shown in FIG. 6 has a first end region e₁′ positional in a first source vein (for example, 68, FIG. 3A; 62, FIG. 3B; 62, FIG. 3C; or, 88, FIG. 5) of the venous furcation (60, FIGS. 3A-3C; or, 80, FIG. 5, respectively), a second end region e₂′ positional in a second source vein (68, FIG. 3C) or in the sink vein (64, FIGS. 3A and 3B; or, 84, FIG. 5, respectively) of the venous furcation (60, FIGS. 3A-3C; or, 80, FIG. 5, respectively), and a middle filtering zone F′ circumferentially and longitudinally extending between first end region e₁′ and second end region e₂′, whereby middle filtering zone F′ of mesh-like element 92 when so positioned in the venous furcation, filters the embolic material (solid circles) from the blood passing through openings or pores 26 of middle filtering zone F′, while substantially not disturbing flow of the blood through the venous furcation, thereby preventing the embolic material from entering the sink vein of the venous furcation in the subject.

As previously stated above, mesh-like element 92 of blood filtering device 90 shown in FIG. 6 has a geometrical configuration or construction characterized by a variable inter-region structural profile in the implanted expanded state, wherein values of dimensional characteristics (ii)-(vi) from region to region of each of the three regions, that is, first end region e₁′, second end region e₂′, and middle filtering zone F′, are notably different, as illustratively described in detail immediately following.

With respect to dimensional characteristic (ii), the values of length, W′, of a side of the opening or pore 26 formed between mesh-like element filaments 24 of each of the three regions of mesh-like element 92, in the implanted expanded state, are in the following relative order: W₁′ of first end region e₁′>W_{F}′ of middle filtering zone F′>W₂′ of second end region e₂′.

With respect to dimensional characteristic (iii), the values of the number of mesh-like element filaments 24, of each of the three regions of mesh-like element 92, are in the following relative order: first end region e₁′<middle filtering zone F′<second end region e₂′.

With respect to dimensional characteristic (iv), the values of angle, α′, between adjacent sides of the non-square or square, parallelogram, shaped, opening or pore 26 formed between crossed or overlapped mesh-like element filaments 24 of each of the three regions of mesh-like element 92, in the implanted expanded state, are in the following relative order: α₁′ and α₂′, the obtuse angle, between 90° and 180°, of first and second end regions e₁′ and e₂′, respectively, >α_{F}′, of 90°, of middle filtering zone F′.

With respect to dimensional characteristic (v), the values of pitch, P′, of turnings of mesh-like element filaments 24 of each of the three regions of mesh-like element 92, in the implanted expanded state, are in the following relative order: P₁′ of first end region e₁′>P_{F}′ of middle filtering zone F′>P₂′ of second end region e₂′.

With respect to dimensional characteristic (vi), the values of the porosity index of each of the three regions of mesh-like element 92, in the implanted expanded state, are in the following relative order: first end region e₁′>middle filtering zone F′>second end region e₂′.

It is especially noted, that for mesh-like element 92 of blood filtering device 90, the smaller value of pitch, P₁′, and the smaller value of the porosity index, of second end region e₂′, compared to the corresponding values of these dimensional characteristics of first end region e₁′, are so selected whereby, with reference and application to FIG. 5, extremity 87 of second end region e₂′ is optimally positional in sink vein 84 at larger diameter, d_{L}, and extremity 85 of first end region e₁′ is optimally positional in source vein 88 at smaller diameter, d_{S}, in venous bifurcation 80.

There is thus a greater overall structural and mechanical strength provided by blood filtering device 90, including an increase of anchoring of mesh-like element 92 to inner wall regions of venous bifurcation 80, compared to mesh-like element 22 of blood filtering device 20. This is a direct result of the previously described phenomenon whereby, in general, decreasing pitch, P, of turnings of mesh-like element filaments 24 in the implanted expanded state, of a particular region or regions, for example, in this case, of second end region e₂, increases the radial force generated by the particular region or regions, that is, second end region e₂, upon the inner wall regions at the respective position or positions, that is, in sink vein 84 at larger diameter, d_{L}, inside venous bifurcation 80.

Simultaneously, the greater value of pitch, P_{F}′, and the greater value of the porosity index, of middle filtering zone F′, compared to the corresponding values of these dimensional characteristics of second end region e₂′, of mesh-like element 92 of blood filtering device 90, are selected so as to advantageously fulfill the previously stated objective of providing an alternative embodiment of blood filtering device 20 which optimally filters the embolic material from the blood passing through pores 26 of middle filtering zone F, and maintaining a deployed implanted expanded position in variable diameter venous bifurcation 80, while substantially not disturbing flow of the blood through venous bifurcation 80.

For the purpose of completeness of description and illustration of the present invention, in a non-limiting manner, first and second end regions e₁′ and e₂′ are shown in FIG. 6 as having the same values of dimensional characteristics of (i), (vii), and (viii), compared to corresponding values of dimensional characteristics (i), (vii), and (viii), of middle filtering zone F′, of mesh-like element 92, as described immediately following.

With respect to dimensional characteristic (i), the value of cross section perimeter, π₁′ and π₂′, of mesh-like element filaments 24, of first and second end regions e₁′ and e₂′, respectively, is the same as the corresponding value of cross section perimeter, π_{F}′, of mesh-like element filaments 24, of middle filtering zone F′. With respect to dimensional characteristic (vii), the value of diameter, D′, of each end region e₁′ and e₂′, respectively, is the same as the corresponding value of diameter, D′, of middle filtering zone F′, of mesh-like element 92 in the implanted expanded state. With respect to dimensional characteristic (viii), the value of luminal length, L₁′ and L₂′, of first and second end regions e₁′ and e₂′, respectively, is the same as the corresponding value of luminal length, L_{F}′, of middle filtering zone F′, of mesh-like element 92 in the implanted expanded state.

Moreover, with respect to the intra-region structural profiles also characterizing the geometrical configuration or construction of mesh-like element 92 of blood filtering device 90, as shown in FIG. 6, values of the entire set of dimensional characteristics (i)-(viii) within each of first and second end regions e₁′ and e₂′, and within middle filtering zone F′, are constant as a function of longitudinal length within each corresponding region along longitudinal axis 94 of mesh-like element 92 in the implanted expanded state.

Another exemplary alternative form of implantable blood filtering device 20 of FIGS. 2A and 2B especially applicable to a variable diameter venous furcation, thereby preventing the above described undesirable potential situation, is herein illustratively described.

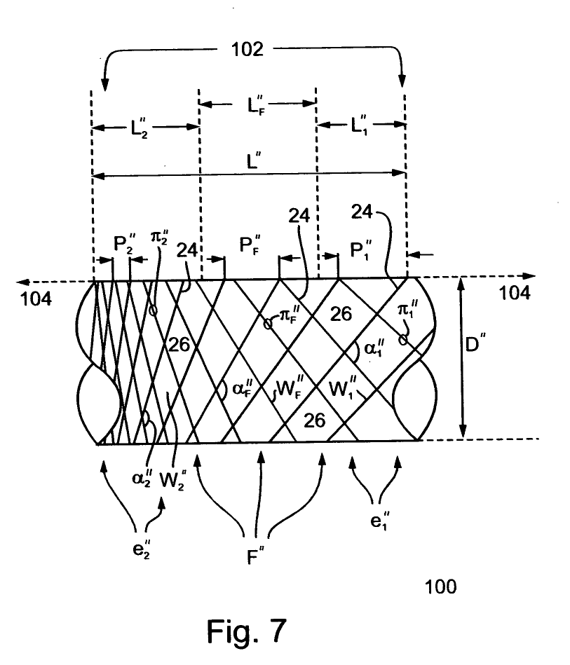

FIG. 7 is a schematic diagram illustrating an exemplary preferred embodiment of a third alternative form of implantable blood filtering device 20 of FIGS. 2A and 2B, herein, for brevity, generally referred to as blood filtering device 100, wherein the geometrical configuration or construction is characterized by a variable inter-region structural profile, whereby values of selected dimensional characteristics (i)-(viii) from region to region of each of the three regions, that is, first end region e₁″, second end region e₂″, and middle filtering zone F″, are notably different, and, is additionally characterized by variable intra-region structural profiles, whereby values of selected dimensional characteristics (i)-(viii) within each of first and second end regions e₁″ and e₂″, and within middle filtering zone F″, vary as a function of longitudinal length within each corresponding region along longitudinal axis 104 of mesh-like element 102 in the implanted expanded state.

The previously described undesirable potential situation relating to a variable diameter venous furcation is prevented by implanting and deploying mesh-like element 102 of blood filtering device 100, which, with reference and application to FIG. 5, enables proper and sufficient anchoring of second end region e₂″ to the larger diameter inner wall region of sink vein 84, in particular, and proper and sufficient anchoring of mesh-like element 102 to the inner wall regions of venous bifurcation 80, in general. Thus, fulfilling the previously stated objective of providing an alternative embodiment of blood filtering device 20 which optimally filters the embolic material from the blood passing through pores 26 of middle filtering zone F, and maintaining a deployed implanted expanded position in variable diameter venous bifurcation 80, while substantially not disturbing flow of the blood through venous bifurcation 80.

As shown in FIG. 7, mesh-like element 102 is geometrically constructed or configured with a variable inter-region structural profile, whereby values of dimensional characteristics (ii), (iii), (iv), (v), and (vi), from region to region of each of the three regions, that is, first end region e₁″, second end region e₂″, and middle filtering zone F″, are notably different, and, is geometrically constructed or configured with variable intra-region structural profiles, whereby values of dimensional characteristics (ii), (iii), (iv), (v), and (vi), within each of first and second end regions e₁″ and e₂″, and within middle filtering zone F″, vary as a function of longitudinal length within each corresponding region along longitudinal axis 104 of mesh-like element 102 in the implanted expanded state.

As for blood filtering device 20, previously described above and illustrated in FIGS. 2A-2B, blood filtering device 100 is an expansible, tubular shaped porous mesh-like element 102, herein, also referred to as mesh-like element 102, formed from mesh-like filaments, fibers, wires, or strands 24. Mesh-like element 102 has openings or pores 26, formed and located in between adjacent mesh-like filaments 24, circumferentially and longitudinally extending along the entirety of mesh-like element 102.

Similar to that previously described above regarding mesh-like element 22 of blood filtering device 20, as illustrated in FIGS. 3A-3C, and, regarding each of mesh-like elements 72 and 92 as illustrated in FIGS. 4 and 6, respectively, here, mesh-like element 102 shown in FIG. 7 has a first end region e₁″ positional in a first source vein (for example, 68, FIG. 3A; 62, FIG. 3B; 62, FIG. 3C; or, 88, FIG. 5) of the venous furcation (60, FIGS. 3A-3C; or, 80, FIG. 5, respectively), a second end region e₂″ positional in a second source vein (68, FIG. 3C) or in the sink vein (64, FIGS. 3A and 3B; or, 84, FIG. 5, respectively) of the venous furcation (60, FIGS. 3A-3C; or, 80, FIG. 5, respectively), and a middle filtering zone F″ circumferentially and longitudinally extending between first end region e₁″ and second end region e₂″, whereby middle filtering zone F″ of mesh-like element 102 when so positioned in the venous furcation, filters the embolic material (solid circles) from the blood passing through openings or pores 26 of middle filtering zone F″, while substantially not disturbing flow of the blood through the venous furcation, thereby preventing the embolic material from entering the sink vein of the venous furcation in the subject.

As previously stated above, mesh-like element 102 of blood filtering device 100 shown in FIG. 7 has a geometrical configuration or construction characterized by a variable inter-region structural profile in the implanted expanded state, wherein values of dimensional characteristics (ii)-(vi) from region to region of each of the three regions, that is, first end region e₁″, second end region e₂″, and middle filtering zone F″, are notably different, as illustratively described in detail immediately following.

With respect to dimensional characteristic (ii), the values of length, W″, of a side of the opening or pore 26 formed between mesh-like element filaments 24 of each of the three regions of mesh-like element 102, in the implanted expanded state, are in the following relative order: W₁″ of first end region e₁″>W_{F}″ of middle filtering zone F″>W₂″ of second end region e₂″.

With respect to dimensional characteristic (iii), the values of the number of mesh-like element filaments 24, of each of the three regions of mesh-like element 102, are in the following relative order: first end region e₁″<middle filtering zone F″<second end region e₂″.

With respect to dimensional characteristic (iv), the values of angle, α″, between adjacent sides of the non-square or square, parallelogram, shaped, opening or pore 26 formed between crossed or overlapped mesh-like element filaments 24 of each of the three regions of mesh-like element 102, in the implanted expanded state, are in the following relative order: α₁″, the obtuse angle, between 90° and 180°, of first end region e₁″<α_{F}″, the obtuse angle, between 90° and 180°, of middle filtering zone F″<α₂″, the obtuse angle, between 90° and 180°, of second end region e₂″.

With respect to dimensional characteristic (v), the values of pitch, P″, of turnings of mesh-like element filaments 24 of each of the three regions of mesh-like element 102, in the implanted expanded state, are in the following relative order: P₁″ of first end region e₁″>P_{F}″ of middle filtering zone F″>P₂″ of second end region e₂″.

With respect to dimensional characteristic (vi), the values of the porosity index of each of the three regions of mesh-like element 102, in the implanted expanded state, are in the following relative order: first end region e₁″>middle filtering zone F″>second end region e₂″.

It is especially noted, that for mesh-like element 102 of blood filtering device 100, the smaller value of pitch, P₁″, and the smaller value of the porosity index, of second end region e₂″, compared to the corresponding values of these dimensional characteristics of first end region e₁″, are so selected whereby, with reference and application to FIG. 5, extremity 87 of second end region e₂″ is optimally positional in sink vein 84 at larger diameter, d_{L}, and extremity 85 of first end region e₁″ is optimally positional in source vein 88 at smaller diameter, d_{S}, in venous bifurcation 80.

There is thus a greater overall structural and mechanical strength provided by blood filtering device 100, including an increase of anchoring of mesh-like element 102 to inner wall regions of venous bifurcation 80, compared to mesh-like element 22 of blood filtering device 20. This is a direct result of the previously described phenomenon whereby, in general, decreasing pitch, P, of turnings of mesh-like element filaments 24 in the implanted expanded state, of a particular region or regions, for example, in this case, of second end region e₂″, increases the radial force generated by the particular region or regions, that is, second end region e₂″, upon the inner wall regions at the respective position or positions, that is, in sink vein 84 at larger diameter, d_{L}, inside venous bifurcation 80.

Simultaneously, the greater value of pitch, P_{F}″, and the greater value of the porosity index, of middle filtering zone F″, compared to the corresponding values of these dimensional characteristics of second end region e₂″, of mesh-like element 102 of blood filtering device 100, are selected so as to advantageously fulfill the previously stated objective of providing an alternative embodiment of blood filtering device 20 which optimally filters the embolic material from the blood passing through pores 26 of middle filtering zone F, and maintaining a deployed implanted expanded position in variable diameter venous bifurcation 80, while substantially not disturbing flow of the blood through venous bifurcation 80.

For the purpose of completeness of description and illustration of the present invention, in a non-limiting manner, first and second end regions e₁″ and e₂″ are shown in FIG. 7 as having the same values of dimensional characteristics of (i), (vii), and (viii), compared to corresponding values of dimensional characteristics (i), (vii), and (viii), of middle filtering zone F″, of mesh-like element 102, as described immediately following.

With respect to dimensional characteristic (i), the value of cross section perimeter, π₁″ and π₂″, of mesh-like element filaments 24, of first and second end regions e₁″ and e₂″, respectively, is the same as the corresponding value of cross section perimeter, π_{F}″, of mesh-like element filaments 24, of middle filtering zone F″. With respect to dimensional characteristic (vii), the value of diameter, D″, of each end region e₁″ and e₂″, respectively, is the same as the corresponding value of diameter, D″, of middle filtering zone F″, of mesh-like element 102 in the implanted expanded state. With respect to dimensional characteristic (viii), the value of luminal length, L₁″ and L₂″, of first and second end regions e₁″ and e₂″, respectively, is the same as the corresponding value of luminal length, L_{F}″, of middle filtering zone F″, of mesh-like element 102 in the implanted expanded state.

Moreover, with respect to the intra-region structural profiles also characterizing the geometrical configuration or construction of mesh-like element 102 of blood filtering device 100, as shown in FIG. 7, values of dimensional characteristics (ii), (iii), (iv), (v), and (vi), within each of first and second end regions e₁″ and e₂″, and within middle filtering zone F″, vary as a function of longitudinal length within each corresponding region along longitudinal axis 104 of mesh-like element 102 in the implanted expanded state. In the specific form of the blood filtering device of the present invention illustrated in FIG. 7, variation of values of dimensional characteristics (ii)-(vi) within each region of the three regions of mesh-like element 102 is particularly illustrated as being continuous as a function of longitudinal length within each corresponding region along longitudinal axis 104 of mesh-like element 102 in the implanted expanded state. Alternatively, variation of values of dimensional characteristics (ii)-(vi) within at least one region of the three regions of mesh-like element 102 is non-continuous or discrete as a function of longitudinal length within the corresponding region along longitudinal axis 104 of mesh-like element 102 in the implanted expanded state.

Another exemplary alternative form of implantable blood filtering device 20 of FIGS. 2A and 2B especially applicable to a variable diameter venous furcation, thereby preventing the previously described undesirable potential situation, is herein illustratively described.

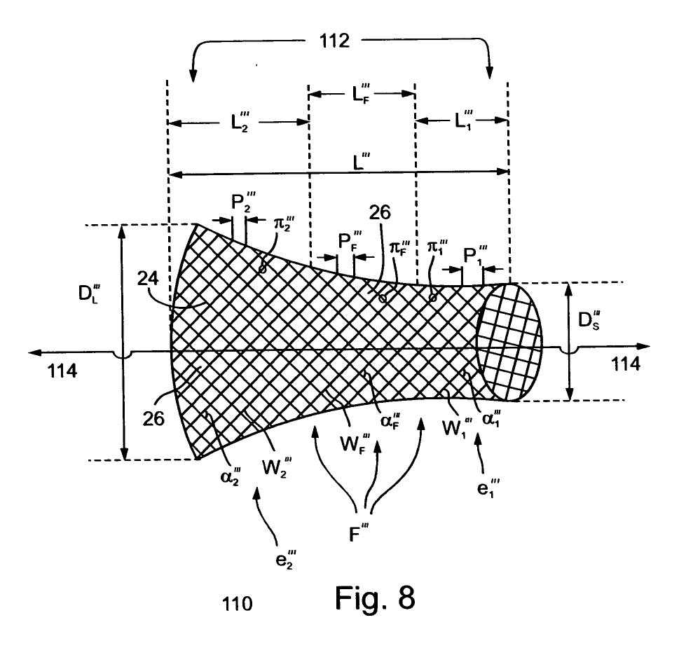

FIG. 8 is a schematic diagram illustrating an exemplary preferred embodiment of a fourth alternative form of implantable blood filtering device 20 of FIGS. 2A and 2B, herein, for brevity, generally referred to as blood filtering device 110, wherein the geometrical configuration or construction is characterized by a variable inter-region structural profile, whereby values of selected dimensional characteristics (i)-(viii) from region to region of each of the three regions, that is, first end region e₁′″, second end region e₂′″, and middle filtering zone F′″, are notably different, and, is additionally characterized by variable intra-region structural profiles, whereby values of selected dimensional characteristics (i)-(viii) within each of first and second end regions e₁′″ and e₂′″, and within middle filtering zone F′″, vary as a function of longitudinal length within each corresponding region along longitudinal axis 114 of mesh-like element 112 in the implanted expanded state.

The previously described undesirable potential situation relating to a variable diameter venous furcation is prevented by implanting and deploying mesh-like element 112 of blood filtering device 110, which, with reference and application to FIG. 5, enables proper and sufficient anchoring of second end region e₂′″ to the larger diameter inner wall region of sink vein 84, in particular, and proper and sufficient anchoring of mesh-like element 112 to the inner wall regions of venous bifurcation 80, in general. Thus, fulfilling the previously stated objective of providing an alternative embodiment of blood filtering device 20 which optimally filters the embolic material from the blood passing through pores 26 of middle filtering zone F, and maintaining a deployed implanted expanded position in variable diameter venous bifurcation 80, while substantially not disturbing flow of the blood through venous bifurcation 80.

As shown in FIG. 8, mesh-like element 112 is geometrically constructed or configured with a variable inter-region structural profile, whereby values of dimensional characteristics (iii) and (vii), from region to region of each of the three regions, that is, first end region e₁′″, second end region e₂′″, and middle filtering zone F′″, are notably different, and, is geometrically constructed or configured with variable intra-region structural profiles, whereby the value of dimensional characteristics (viii), within each of first and second end regions e₁′″ and e₂′″, and within middle filtering zone F′″, varies as a function of longitudinal length within each corresponding region along longitudinal axis 114 of mesh-like element 112 in the implanted expanded state.

As for blood filtering device 20, previously described above and illustrated in FIGS. 2A-2B, blood filtering device 110 is an expansible, tubular shaped porous mesh-like element 112, herein, also referred to as mesh-like element 112, formed from mesh-like filaments, fibers, wires, or strands 24. Mesh-like element 112 has openings or pores 26, formed and located in between adjacent mesh-like filaments 24, circumferentially and longitudinally extending along the entirety of mesh-like element 112.

Mesh-like element 112 is particularly geometrically constructed or configured with a cone-like, bulbous, or semi-hyperboloidal kind of tubular shape, whereby mesh-like element 112 circumferentially flares, that is, radially outwardly expands along a longitudinal axis, for example, longitudinal axis 114, of mesh-like element 112, from the opening at the extremity or end of first end region e₁′″ to the opening at the extremity or end of second end region e₂′″. Accordingly, the value of dimensional characteristic (vii) diameter, D′″, of mesh-like element 112 in the implanted expanded state, increases along a longitudinal axis, for example, longitudinal axis 114, of mesh-like element 112, from the diameter, D_{S}′″, of the opening at the extremity or end of first end region e₁′″ to the diameter, D_{L}′″, of the opening at the extremity or end of second end region e₂′″, as illustrated in FIG. 8.

Similar to that previously described above regarding mesh-like element 22 of blood filtering device 20, as illustrated in FIGS. 3A-3C, and, regarding each of mesh-like elements 72, 92, and 102, as illustrated in FIGS. 4, 6, and 7, respectively, here, mesh-like element 112 shown in FIG. 8 has a first end region e₁′″ positional in a first source vein (for example, 68, FIG. 3A; 62, FIG. 3B; 62, FIG. 3C; or, 88, FIG. 5) of the venous furcation (60, FIGS. 3A-3C; or, 80, FIG. 5, respectively), a second end region e₂′″ positional in a second source vein (68, FIG. 3C) or in the sink vein (64, FIGS. 3A and 3B; or, 84, FIG. 5, respectively) of the venous furcation (60, FIGS. 3A-3C; or, 80, FIG. 5, respectively), and a middle filtering zone F′″ circumferentially and longitudinally extending between first end region e₁′″ and second end region e₂′″, whereby middle filtering zone F′″ of mesh-like element 112 when so positioned in the venous furcation, filters the embolic material (solid circles) from the blood passing through openings or pores 26 of middle filtering zone F′″, while substantially not disturbing flow of the blood through the venous furcation, thereby preventing the embolic material from entering the sink vein of the venous furcation in the subject.

As previously stated above, mesh-like element 112 of blood filtering device 110 shown in FIG. 8 has a geometrical configuration or construction characterized by a variable inter-region structural profile in the implanted expanded state, wherein values of dimensional characteristics (iii) and (vii) from region to region of each of the three regions, that is, first end region e₁′″, second end region e₂′″, and middle filtering zone F′″, are notably different, as illustratively described in detail immediately following.

With respect to dimensional characteristic (iii), the values of the number of mesh-like element filaments 24, of each of the three regions of mesh-like element 112, are in the following relative order: first end region e₁′″<middle filtering zone F′″<second end region e₂′″.

With respect to dimensional characteristic (vii), the values of diameter, D′″, of each of the three regions of mesh-like element 112, in the implanted expanded state, are in the following relative order: D₁′″ of first end region e₁″<D_{F}′″ of middle filtering zone F′″<D₂′″ of second end region e₂′″.

It is especially noted, that for mesh-like element 112 of blood filtering device 110, the smaller values of diameter, D₁′″, of first end region e₁′″, compared to the values of the diameter, D₁′″, of second end region e₂′″, are so selected whereby, with reference and application to FIG. 5, extremity 87 of second end region e₂′″ is optimally positional in sink vein 84 at larger diameter, d_{L}, and extremity 85 of first end region e₁′″ is optimally positional in source vein 88 at smaller diameter, d_{S}, in venous bifurcation 80.

There is thus a greater overall structural and mechanical strength provided by blood filtering device 110, including an increase of anchoring of mesh-like element 112 to inner wall regions of venous bifurcation 80, compared to mesh-like element 22 of blood filtering device 20. This is a direct result of a larger radial force generated by the particular region, that is, second end region e₂′″, upon the inner wall regions at the respective position, that is, in sink vein 84 at larger diameter, d_{L}, inside venous bifurcation 80.

For the purpose of completeness of description and illustration of the present invention, in a non-limiting manner, first and second end regions e₁′″ and e₂′″ are shown in FIG. 8 as having the same values of dimensional characteristics of (i), (ii), (iv), (v), (vi), and (viii), compared to corresponding values of dimensional characteristics (i), (ii), (iv), (v), (vi), and (viii), of middle filtering zone F′″, of mesh-like element 112, as described immediately following.

With respect to dimensional characteristic (i), the value of cross section perimeter, π₁′″ and π₂′″, of mesh-like element filaments 24, of first and second end regions e₁′″ and e₂′″, respectively, is the same as the corresponding value of cross section perimeter, π_{F}′″, of mesh-like element filaments 24, of middle filtering zone F′″.

With respect to dimensional characteristic (ii), the value of length, W₁′″ and W₂′″, of a side of opening or pore 26 formed between mesh-like element filaments 24 of first and second end regions e₁′″ and e₂′″, respectively, is the same as the corresponding value of length, W_{F}′″, of middle filtering zone F′″, of mesh-like element 112 in the implanted expanded state.

With respect to dimensional characteristic (iv), the value of angle, α₁′″ and α₂′″, of 90°, between adjacent sides of the square shaped opening or pore 26 formed between crossed or overlapped mesh-like element filaments 24 of first and second end regions e₁′″ and e₂′″, respectively, is the same as the corresponding value of angle, α_{F}′″, of 90°, of middle filtering zone F′″, of mesh-like element 112 in the implanted expanded state.

With respect to dimensional characteristic (v), the value of pitch, P₁′″ and P₂′″, of turnings of mesh-like element filaments 24, of first and second end regions e₁′″ and e₂′″, respectively, is the same as the corresponding value of pitch, P_{F}′″, of middle filtering zone F′″, of mesh-like element 112 in the implanted expanded state.

With respect to dimensional characteristic (vi), the value of the porosity index of first and second end regions e₁′″ and e₂′″, respectively, is the same as the corresponding value of the porosity index of middle filtering zone F′″, of mesh-like element 112 in the implanted expanded state.

With respect to dimensional characteristic (viii), the value of luminal length, L₁′″ and L₂′″, of first and second end regions e₁′″ and e₂′″, respectively, is the same as the corresponding value of luminal length, L_{F}′″, of middle filtering zone F′″, of mesh-like element 112 in the implanted expanded state.

Moreover, with respect to the intra-region structural profile also characterizing the geometrical configuration or construction of mesh-like element 112 of blood filtering device 110, as previously described above and shown in FIG. 8, due to the geometrical configuration or construction of mesh-like element 112 being a cone-like or semi-hyperboloidal kind of tubular shape, whereby mesh-like element 112 circumferentially flares, that is, radially outwardly expands along a longitudinal axis, for example, longitudinal axis 114, of mesh-like element 112, from the opening at the extremity or end of first end region e₁′″ to the opening at the extremity or end of second end region e₂′″, the value of dimensional characteristic (vii), diameter, D′″, within each of first and second end regions e₁′″ and e₂′″, and within middle filtering zone F′″, varies as a function of longitudinal length within each corresponding region along longitudinal axis 114 of mesh-like element 112 in the implanted expanded state.

In the specific form of the blood filtering device of the present invention illustrated in FIG. 8, variation of the value of dimensional characteristic (vii), diameter, D′″, within each region of the three regions of mesh-like element 112 is particularly illustrated as being continuous as a function of longitudinal length within each corresponding region along longitudinal axis 114 of mesh-like element 112 in the implanted expanded state. Alternatively, variation of the value of dimensional characteristic (vii), diameter, D′″, within at least one region of the three regions of mesh-like element 112 is non-continuous or discrete as a function of longitudinal length within each corresponding region along longitudinal axis 114 of mesh-like element 112 in the implanted expanded state.

Another exemplary alternative form of implantable blood filtering device 20 of FIGS. 2A and 2B especially applicable to a variable diameter venous furcation, is directly related to, and an extension of, previously described exemplary preferred embodiment of a fourth alternative form of implantable blood filtering device 20 of FIGS. 2A and 2B, that is, blood filtering device 110 illustrated in FIG. 8.

For this additional exemplary alternative form, not illustrated herein, but referring to mesh-like element 112 of blood filtering device 110 illustrated in FIG. 8, the mesh-like element is particularly geometrically constructed or configured with a double cone-like, double bulbous, or full hyperboloidal kind of tubular shape, wherein mesh-like element 112 is appropriately geometrically ‘copied, oppositely matched and connected’ to itself at middle filtering zone F′″, whereby the mesh-like element circumferentially flares, that is, radially outwardly expands along a longitudinal axis, such as longitudinal axis 114 of mesh-like element 112, from the first extremity or end of the, longer, middle filtering zone F′″ to the opening at the extremity or end of first end region e₁′″, and, from the second extremity or end of middle filtering zone F′″ to the opening at the extremity or end of second end region e₂′″. Accordingly, the value of dimensional characteristic (vii) diameter, D′″, of such a mesh-like element in the implanted expanded state, increases along a longitudinal axis, such as longitudinal axis 114 of mesh-like element 112, from the diameter, D_{S}′″, at the center of middle filtering zone F′″, to the diameter, D_{L}′″, of the opening at the extremity or end of each first and second end region e₁′″ and e₂′″. Implementing, that is, inserting, positioning, implanting, and deploying such a double cone-like, double bulbous, or full hyperboloidal kind of expansible, tubular shaped porous mesh-like element is similar to those procedures for implementing previously described mesh-like element 112 of blood filtering device 110.

The expansible, tubular shaped porous mesh-like element, that is, mesh-like element 22 (FIGS. 2A-2B), or, an alternative embodiment or form thereof, such as mesh-like element 72, 92, 102, or 112 (FIG. 4, 6, 7, or 8, respectively), in general, and, mesh-like element filaments, fibers, wires, or strands 24, in particular, of the blood filtering device, that is, blood filtering device 20 (FIGS. 2A-2B), or, an alternative embodiment or form thereof, such as blood filtering device 70, 90, 100, or 110 (FIG. 4, 6, 7, or 8, respectively), of the present invention, are made of a material having an elasticity suitable for expanding from a contracted position in which it is inserted into the vascular system of a subject, and expanded by means well known in the art, for treating and/or preventing a condition associated with embolic material in blood flowing from at least one source vein towards and into the sink vein of a venous furcation in a subject, as further illustratively described herein below.

Mesh-like element filaments, fibers, wires, or strands 24 are made of any suitable material which is bio-compatible and which can be worked, that is, braided, plaited, interwoven, interweaved, woven, weaved, interlaced, or knitted, into an expansible, tubular shaped porous mesh-like element, and processed to retain the previously described geometrical configuration or construction characterized by two types of structural profiles of (1) an ‘inter-region’ structural profile and (2) ‘intra-region’ structural profiles, determined by a combination of critical ranges of values of the previously described dimensional characteristics (i)-(viii), for optimally filtering the embolic material from the blood passing through pores of the middle filtering zone of the mesh-like element, and maintaining a deployed implanted expanded position in the venous furcation, while substantially not disturbing flow of the blood through the venous furcation, thereby highly effectively preventing the embolic material from entering the sink vein of the venous furcation and from migrating downstream therefrom in the circulatory system of the subject. Bio-compatible material refers to any material that can be safely introduced and implanted in a human or animal subject for an indefinite period of time without causing undesirable physiological damage or pain to the subject.

More specifically, mesh-like element filaments, fibers, wires, or strands 24 are made of a material selected from the group consisting of stainless steel, for example, 316L stainless steel, tantalum, cobalt base alloy, nitinol, superelastic nitinol, shaped memory alloy, polymeric material, and, combinations thereof.

Optionally, each of a number of, or all of, mesh-like element filaments, fibers, wires, or strands 24, made of at least one of the previously indicated materials, are clad with a cladding, that is, a metal coating, covering, or sheathing, bonded onto the indicated material. Optionally, each of a number of, or all of, mesh-like element filaments, fibers, wires, or strands 24, made of at least one of the previously indicated materials, are coated or covered with a bio-compatible coating or covering, as described by Ulrich Sigwart, in “Endoluminal Stenting”, W.B. Saunders Company Ltd., London 1996. Optionally, each of a number of, or all of, mesh-like element filaments, fibers, wires, or strands 24, made of at least one of the previously indicated materials, are coated or covered with a biological and/or pharmaceutical coating or covering, for example, a coating or covering being or including a drug, whereby the drug is either an immediate time release type of drug or a delayed time release type of drug.

As previously stated above, the geometrical shape or form of the cross section of mesh-like element filaments, fibers, wires, or strands 24 is preferably circular or round, but, in a non-limiting manner, may also be elliptical, square, or rectangular.

As previously indicated, herein, the term ‘mesh-like’ is used throughout the disclosure as a descriptor for further describing and clarifying the geometrical configuration or construction of the expansible, tubular shaped porous element of the implantable blood filtering device, and alternative embodiments thereof, of the present invention. In the context of the present invention, the term ‘mesh-like’ denotes a net or network of crossed or overlapped filaments, fibers, wires, or strands, used for configuring or constructing the expansible, tubular shaped porous mesh-like element of the implantable blood filtering device, and alternative embodiments thereof, of the present invention. It is to be fully understood that the term ‘mesh-like’ generally refers to synonymous, directly related, alternative, and/or more specific or limiting descriptors such as, but not limited to, braided, plaited, interwoven, interweaved, woven, weaved, interlaced, and knitted, whereby each of these terms may equivalently, relatedly, alternatively, or more specifically, be used as an appropriate descriptor for further describing and clarifying the geometrical configuration or construction of the expansible, tubular shaped porous mesh-like element of the implantable blood filtering device, and alternative embodiments thereof, of the present invention.

Preferably, the expansible, tubular shaped porous mesh-like element, and alternative embodiments thereof, are braided, however, as previously stated, the expansible, tubular shaped porous mesh-like element, and alternative embodiments thereof, are each of a directly related, alternative, and/or more specific or limiting geometrical configuration or construction, selected from the group consisting of plaited, interwoven, interweaved, woven, weaved, interlaced, and knitted.

Mesh-like element filaments 24 are meshed, in general, and braided, in particular, according to any technique known in the art of meshing, in general, and braiding, in particular, tubular shaped porous elements or bodies, for example, as described in U.S. Pat. No. 4,655,771, issued to Wallsten, the description of which is incorporated by reference as if fully set forth herein.

The blood filtering device of the present invention, or any alternative embodiment or form thereof, is constructed in a way very similar to conventional stents. In brief, typically, the mesh-like element is produced by combining one or more filament, fiber, wire, or strand material, each of which passes over and under one or more other or same filament, fiber, wire, or strand material in a meshed manner, in general, and in a braided manner, in particular, as they are wound about a cylinder, cone, or contoured mandrel, according to the previously described geometrical configuration or construction characterized by the two types of structural profiles, featuring constant or variable dimensional characteristics (i)-(viii). The precursor mesh-like structure of the mesh-like element is cut, for example, by laser cutting, through circumferential cross sections separated by desired luminal lengths, L, for forming the mesh-like element of the present invention. The mesh-like element is removed from the cylinder, cone, or contoured mandrel, during or after processing.

After meshing, in general, or braiding, in particular, is completed, it is desirable, but not necessary, to anneal the formed mesh-like element configuration or structure. Thermal annealing is preferred, which is performed at a temperature and for a period of time appropriate to the selected material. For example, for nitinol as the material of the mesh-like element, thermal annealing is performed at a temperature of about 500° C., for about 10 minutes. Additional finishing processes, such as polishing, may be required, depending on the type of filament, fiber, wire, or strand, material and the particular manufacturing method.

Preferably, the expansible, tubular shaped porous mesh-like element, of the blood filtering device of the present invention, is configured or constructed by employing a meshing technique, in general, or braiding technique, in particular, such as just described above. Alternatively, the mesh-like element is constructed using well known techniques of photochemical engraving, or, another etching process, applicable for forming a mesh-like element, such as that described herein above. Any such technique is used for configuring or constructing the mesh-like element, as long as the completely formed and functional mesh-like element has the previously described geometrical configuration or construction characterized by the two types of structural profiles of (1) an ‘inter-region’ structural profile and (2) ‘intra-region’ structural profiles, determined by a combination of critical ranges of values of the previously described dimensional characteristics (i)-(viii), and whereby the mesh-like element is sufficiently flexible; can be compressed for introduction into the venous system of a subject; and, when it radially expands, it exerts sufficient force against sides of blood vessels for self-anchoring to the blood vessels, as previously described above.

A first specific example is provided herein, for briefly describing configuration or construction of previously described mesh-like element 72 of blood filtering device 70, as illustrated in FIG. 4, featuring middle filtering zone F′ and first and second end regions e₁′ and e₂′, wherein, with respect to the previously described inter-region structural profile, relating to comparison among specific geometrical configurations or constructions of the three regions of mesh-like element 72, first and second end regions e₁′ and e₂′ have different values of dimensional characteristics of (ii), (iii), (iv), (v), and (vi), compared to, that is, greater than or less than, corresponding values of dimensional characteristics (ii), (iii), (iv), (v), and (vi), of middle filtering zone F′, of mesh-like element 72.

Mesh-like element 72 is configured or constructed by using appropriate prior art techniques and equipment for cutting and welding or soldering such types of mesh-like forms. For example, construction of mesh-like element 72 is done by starting with mesh-like element 20 (FIG. 2A) and cutting, for example, by laser cutting, mesh-like element 20 through two circumferential cross sections along longitudinal axis 44 which are separated by a desired luminal length, L_{F}, of middle filtering zone F, subsequently corresponding to luminal length, L_{F}′, of middle filtering zone F′. Then, there is soldering or welding, for example, by laser soldering or welding, the extremities or ends of geometrically configured middle filtering zone F′ to first and second end regions e₁ and e₂ of mesh-like element 20.

A second specific example is provided herein, for briefly describing configuration or construction of previously described mesh-like element 112 of blood filtering device 110, as illustrated in FIG. 8, which is particularly geometrically constructed or configured with a cone-like or semi-hyperboloidal kind of tubular shape, whereby mesh-like element 112 circumferentially flares, that is, radially outwardly expands along a longitudinal axis, for example, longitudinal axis 114, of mesh-like element 112, from the opening at the extremity or end of first end region e₁′″ to the opening at the extremity or end of second end region e₂′″. Mesh-like element 112 is configured or constructed by using appropriate prior art techniques and equipment for meshing, in general, and braiding, in particular, and cutting such types of mesh-like forms, for example, involving the use of a cone-like, bulbous, or semi-hyperboloidal type of contoured mandrel having an appropriately enlarged, flared, or bulbous shaped end.

It is briefly noted herein, that the expansible, tubular shaped porous mesh-like element, that is, mesh-like element 22 (FIGS. 2A-2B), or, an alternative embodiment or form thereof, such as mesh-like element 72, 92, 102, or 112 (FIG. 4, 6, 7, or 8, respectively), of the blood filtering device, that is, blood filtering device 20 (FIGS. 2A-2B), or, an alternative embodiment or form thereof, such as blood filtering device 70, 90, 100, or 110 (FIG. 4, 6, 7, or 8, respectively), of the present invention, does not necessarily need to be self-expansible. Accordingly, the mesh-like element may be made of a non-self-expansible mesh-like material, that is expansible under pressure supplied by a separate implantable deploying device or mechanism, such as by an implantable expansible balloon. In this case, deployment of the mesh-like element of the blood filtering device is carried out as for conventional stents, by placing the mesh-like element of the blood filtering device in a compressed or contracted state around the expansible balloon, followed by controllably expanding the balloon under pressure once the mesh-like element of the blood filtering device reaches the desired location and placed according to the desired positioning.

Further description of the corresponding method for filtering embolic material from blood flowing from at least one source vein into the sink vein of a venous furcation in a subject, utilizing blood filtering device 20 (FIGS. 2A-2B), or, an alternative embodiment or form thereof, such as blood filtering device 70, 90, 100, or 110 (FIG. 4, 6, 7, or 8, respectively), according to the present invention, is provided herein.

In the following description of the method of the present invention, included are only main or principal steps needed for sufficiently understanding proper ‘enabling’ utilization and implementation of the disclosed implantable blood filtering device. Accordingly, descriptions of the various required or optional minor, intermediate, and/or, sub steps, which are readily known by one of ordinary skill in the art, and/or, which are available in the prior art and technical literature relating to inserting, implanting, positioning, and deploying implantable, intravascular or intraluminal tubular mesh-like devices, such as braided stents, are not included herein.

In Step (a) of the method for filtering embolic material from blood flowing from at least one source vein into the sink vein of a venous furcation in a subject, there is providing implantable blood filtering device 20 (FIGS. 2A-2B), or, an alternative embodiment or form thereof, such as blood filtering device 70, 90, 100, or 110 (FIG. 4, 6, 7, or 8, respectively), as previously described and illustrated above, being an expansible, tubular shaped porous mesh-like element 22 (FIGS. 2A-2B), or, an alternative embodiment or form thereof, such as mesh-like element 72, 92, 102, or 112 (FIG. 4, 6, 7, or 8, respectively), having a first end region positional in a first source vein (for example, 68, FIG. 3A; 62, FIG. 3B; 62, FIG. 3C; or, 88, FIG. 5) of the venous furcation (for example, 60, FIGS. 3A-3C; or, 80, FIG. 5, respectively), a second end region positional in a second source vein (for example, 68, FIG. 3C) or in the sink vein (for example, 64, FIGS. 3A and 3B; or, 84, FIG. 5, respectively) of the venous furcation (60, FIGS. 3A-3C; or, 80, FIG. 5, respectively), and a middle filtering zone circumferentially and longitudinally extending between the first end region and the second end region.

In Step (b), there is implanting and deploying the implantable blood filtering device of Step (a) in the venous furcation, whereby the middle filtering zone of the mesh-like element when so positioned in the venous furcation, filters the embolic material from the blood passing through openings or pores of the middle filtering zone, while substantially not disturbing flow of the blood through the venous furcation, thereby preventing the embolic material from entering the sink vein of the venous furcation in the subject.

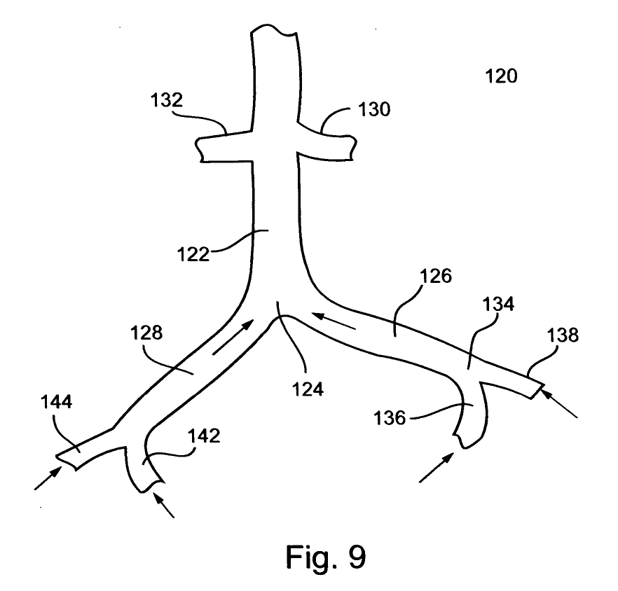

FIG. 9 is a schematic diagram illustrating exemplary venous bifurcation types of venous furcations in the circulatory system of a subject, applicable to deploying the above described and illustrated exemplary preferred embodiments of the implantable blood filtering device, according to the previously described alternative types of deployment illustrated in FIGS. 3A-3C, and in FIG. 5, and in accordance with above Steps (a) and (b).

In FIG. 9, 120 depicts the venous bifurcation of the inferior vena cava vein, including sink vein 122 which splits or bifurcates, at bifurcation point 124, into source veins 126 and 128, known as the right and left common iliac veins, respectively. 130 and 132 are the right and left renal veins, respectively. Source vein, right common iliac vein 126 also serves as a sink vein of another venous bifurcation which splits or bifurcates, at bifurcation point 134, into source veins 136 and 138, known as the internal and external iliac veins, respectively. Source vein, left common iliac vein 128 also serves as a sink vein of another venous bifurcation which splits or bifurcates, at bifurcation point 140, into source veins 142 and 144, also known as internal and external iliac veins, respectively.

Accordingly, with reference to FIG. 9, the implantable blood filtering device of the present invention is deployed and operates at any one of the indicated venous bifurcation points, that is, at any one of venous bifurcation points 124, 134, or 140, whereby the direction of the blood flowing at the venous bifurcation point is from and through each of the indicated two source veins toward and into the indicated sink vein of the corresponding venous bifurcation. In FIG. 9, arrows show a known or anticipated direction of travel of embolic material (not shown) in the blood flowing from at least one of the indicated source veins towards and into the sink vein of the corresponding venous bifurcation. The known or anticipated direction of travel of the embolic material in the flowing blood is used to determine where most effectively to implant and deploy the blood filtering device, according to a particular clinical situation.

With reference to FIG. 9, a first exemplary specific application of the present invention is whereby the blood filtering device filters embolic material from blood flowing from and through right and/or left common iliac veins (source veins) 126 and/or 128, respectively, towards and into inferior vena cava vein (sink vein) 122 of inferior vena cava vein bifurcation 120, thereby preventing the embolic material from entering inferior vena cava vein (sink vein) 122 and from migrating downstream therefrom in the circulatory system of the subject.

A second exemplary specific application of the present invention is whereby the blood filtering device filters embolic material from blood flowing from and through internal and/or external iliac veins (source veins) 136 and 138, respectively, towards and into right common iliac vein (sink vein) 126 of common iliac vein bifurcation 134, thereby preventing the embolic material from entering right common iliac vein (sink vein) 126 and from migrating downstream therefrom in the circulatory system of the subject.

Introduction of the implantable blood filtering device of the present invention into the vascular system, guiding it to and implanting it at a desired location, positioning it, and its deployment, in a venous furcation, are accomplished by using standard equipment and techniques. These techniques, including solutions to the problem of radioopacity of the very thin mesh-like element filaments 24 used for configuring and constructing the blood filtering device, as well as delivery and deployment equipment and systems are extensively discussed in PCT International Publication No. WO 02/0579, published Jan. 24, 2002, of PCT Application No. PCT/IL01/00624, entitled: “Implantable Braided Stroke Preventing Device And Method Of Manufacturing”, and also in PCT Patent Application No. PCT/IL02/00023, entitled: “System And Corresponding Method For Deploying An Implantable Expansible Intraluminal Device”, each by the same applicant of the present disclosure.

As previously described above, for introduction into the vascular system of a subject, the mesh-like element of the blood filtering device is radially compressed and elongates, whereby luminal length, L, of the mesh-like element in the contracted state, is longer than that in the implanted expanded state by an amount in the range of between about 50% to about 500%, corresponding to the luminal length, L, of the mesh-like element in the contracted state, having a value in the range of between about 24 mm to about 500 mm. Introduction of the mesh-like element in the contracted state into the vascular system of a subject may be performed using a 4-5 French catheter.

If desired, an expansible balloon (not shown herein) can be used for assisting deployment of either a self-expansible or a non-self-expansible embodiment of the blood filtering device, and especially for assisting in bringing end regions e₁ and e₂ of the mesh-like element of the blood filtering device into firm contact with the inner wall regions of the source and/or sink veins of the venous furcation in which it is placed. When using a balloon to assist in deploying the blood filtering device, it is desirable to make use of a balloon that expands from the distal end progressively towards the proximal end. In this manner, the blood filtering device is thereby held against the inner wall regions of the veins at the start of the expansion process, whereby the correct positioning of the mesh-like element is assured as the luminal length, L, of the mesh-like element shortens significantly while expanding to the operative deployed implanted expanded state.

Additional aspects of implementing the corresponding method for filtering embolic material from blood flowing from at least one source vein into the sink vein of a venous furcation in a subject, utilizing the implantable blood filtering device, described herein above, according to the present, are provided herein. It is to be fully understood that the following alternative methods of the present invention are each implemented by using implantable blood filtering device 20 (FIGS. 2A-2B), or, an alternative embodiment or form thereof, such as blood filtering device 70, 90, 100, or 110 (FIG. 4, 6, 7, or 8, respectively), as previously described and illustrated above.

The method for preventing and/or treating the occurrence of a condition associated with embolic material in blood flowing from at least one source vein into the sink vein of a venous furcation in a subject, features the steps of: (a) providing an implantable blood filtering device comprising an expansible, tubular shaped porous mesh-like element of filaments, having a first end region positional in a first source vein of the venous furcation, a second end region positional in a second source vein or in the sink vein of the venous furcation, and a middle filtering zone circumferentially and longitudinally extending between the first and second end regions; and (b) implanting and deploying the implantable blood filtering device in the venous furcation, whereby the middle filtering zone of the mesh-like element when so positioned in the venous furcation, filters the embolic material from the blood passing through pores of the middle filtering zone, while substantially not disturbing flow of the blood through the venous furcation, thereby preventing the embolic material from entering the sink vein of the venous furcation of the subject.

The use of an implantable blood filtering device in the manufacture of a medical device for preventing and/or treating the occurrence of a condition associated with embolic material in blood flowing from at least one source vein into the sink vein of a venous furcation in a subject, features the steps of: (a) providing the implantable blood filtering device comprising an expansible, tubular shaped porous mesh-like element of filaments, having a first end region positional in a first source vein of the venous furcation, a second end region positional in a second source vein or in the sink vein of the venous furcation, and a middle filtering zone circumferentially and longitudinally extending between the first and second end regions; and (b) implanting and deploying the implantable blood filtering device in the venous furcation, whereby the middle filtering zone of the mesh-like element when so positioned in the venous furcation, filters the embolic material from the blood passing through pores of the middle filtering zone, while substantially not disturbing flow of the blood through the venous furcation, thereby preventing the embolic material from entering the sink vein of the venous furcation of the subject.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the spirit and broad scope of the appended claims.

1. An implantable blood filtering device for implantation in a venous furcation of two source veins into a sink vein to filter embolic material from the blood in one of said source veins before flowing into said sink vein;
said device comprising a tubular-shaped porous structure having a first end region configured and dimensioned for anchoring in one of said veins at said venous furcation; a second end region configured and dimensioned for anchoring in another of said veins at said venous furcation; and a middle filtering zone between said first and second end regions;
said middle filtering zone having pores configured and dimensioned so as to be effective, when said first and second end regions of the tubular-shaped porous structure are anchored in their respective veins, to filter the embolic material in the blood flowing in said one source vein before entering said sink vein.

2. The device according to claim 1, wherein said tubular-shaped porous structure is configured and dimensioned for anchoring said first end region in one of said source veins, and said second end region in said sink vein.

3. The device according to claim 1, wherein said tubular-shaped porous structure is configured and dimensioned for anchoring said first and second end regions in said two source veins.

4. The device according to claim 1, wherein the length (W) of each side of a pore in said middle filtering zone, in the implanted condition of the device, is 0.3-7 mm.

5. The device according to claim 1, wherein the length (W) of each side of a pore in said middle filtering zone, in the implanted condition of the device, is 2-3 mm.

6. The device according to claim 1, wherein said middle filtering zone, in the implanted condition of the device, has a porosity index (PI) of 50-95%.

7. The device according to claim 1, wherein said anchoring in one of said veins and said anchoring in said another of said veins is accomplished by radial force.

8. The device according to claim 1, wherein said tubular-shaped porous structure is of a mesh-like construction, having a plurality of openings, said tubular-shaped porous structure being capable of having a small-diameter contracted state to facilitate its delivery to the venous furcation via a catheter, and a large-diameter expanded state for implanting in said venous furcation.

9. The device according to claim 1, wherein said tubular-shaped porous structure is constructed of a plurality of interwoven filaments.

10. The device according to claim 9, wherein the cross-section perimeter (π) of said filaments in said middle filtering zone is 80-2500 μm.

11. The device according to claim 9, wherein the cross-section perimeter (π) of said filaments in said middle filtering zone is 180-1300 μm

12. The device according to claim 9, wherein the number of filaments (n) in said middle filtering zone is 6-92.

13. The device according to claim 9, wherein the filaments in said middle filtering zone, in the implanted condition of the device, form an angle (α) of 95-140° with respect to each other.

14. The device according to claim 9, wherein the filaments in said middle filtering zone, in the implanted condition of the device, define a pitch (p) of 0.5-10 mm.

15. The device according to claim 9, wherein the filaments in said first end region define a first pitch; the filaments in said second end region define a second pitch; and the filaments in said middle filtering zone define a third pitch, wherein said first pitch and said second pitch are each less than said third pitch.

16. A method of filtering embolic material in a source vein from flowing into a sink vein at a venous furcation of said sink vein with said source vein and at least one other source vein, comprising:
providing an implantable blood filtering device according to claim 1; and
implanting said filtering device into said venous furcation.

17. The method according to claim 16, wherein said tubular-shaped porous structure is configured and dimensioned for anchoring said first end region in one of said source veins, and said second end region in said sink vein.

18. The method according to claim 16, wherein said tubular-shaped porous structure is configured and dimensioned for anchoring said first and second end regions in said two source veins.

19. The method according to claim 16, wherein the length (W) of each side of a pore in said middle filtering zone, in the implanted condition of the device, is 0.3-7 mm.

20. The method according to claim 16, wherein the length (W) of each side of a pore in said middle filtering zone, in the implanted condition of the device, is 2-3 mm.

21. The method according to claim 16, wherein said middle filtering zone, in the implanted condition of the device, has a porosity index (PI) of 50-95%.

22. The method according to claim 16, wherein said anchoring in one of said veins and said anchoring in said another of said veins is accomplished by radial force.

23. The method according to claim 16, wherein said tubular-shaped porous structure is of a mesh-like construction, having a plurality of openings, said tubular-shaped porous structure being capable of having a small-diameter contracted state to facilitate its delivery to the venous furcation via a catheter, and a large-diameter expanded state for implanting in said venous furcation.

24. The method according to claim 16, wherein said tubular-shaped porous structure is constructed of a plurality of interwoven filaments.

25. The method according to claim 24, wherein the cross-section perimeter (π) of said filaments in said middle filtering zone is 80-400 μm.

26. The method according to claim 24, wherein the cross-section perimeter (π) of said filaments in said middle filtering zone is 80-2500 μm.

27. The method according to claim 24, wherein the number of filaments (n) in said middle filtering zone is 6-92.

28. The method according to claim 24, wherein the filaments in said middle filtering zone, in the implanted condition of the device, form an angle (α) of 95-140° with respect to each other.

29. The method according to claim 24, wherein the filaments in said middle filtering zone, in the implanted condition of the device, define a pitch (p) of 0.5-10 mm.

30. The method according to claim 24, wherein the filaments in said first end region define a first pitch; the filaments in said second end region define a second pitch; and the filaments in said middle filtering zone define a third pitch, wherein said first pitch and said second pitch are each less than said third pitch.
